(19) United States
(12) Patent Application Publication
(10) Pub. No.: US 2024/0335792 A1
(43) Pub. Date: Oct. 10, 2024

(54) METHOD FOR EXTRACTING EXOSOME

(71) Applicant: GUANGZHOU ZHAORUI MEDICAL BIOTECHNOLOGY CO, LTD, Guangzhou (CN)

(72) Inventors: Zhenyu XU, Guangzhou (CN); Xiaodong Lin, Guangzhou (CN); Yingna Chen, Guangzhou (CN); Dishen Chen, Guangzhou (CN)

(21) Appl. No.: 18/749,708

(22) Filed: Jun. 21, 2024

Related U.S. Application Data
(63) Continuation of application No. PCT/CN2022/082583, filed on Mar. 23, 2022.

(30) Foreign Application Priority Data
Dec. 24, 2021 (CN) 202111602970.5

(51) Int. Cl. B01D 61/14; B01D 61/58; B01D 71/12; B01D 71/68

(52) U.S. Cl. CPC B01D 61/147; B01D 61/58; B01D 71/12; B01D 71/68; B01D 2323/216; B01D 2325/36

(57) ABSTRACT

The present application relates to the technical field of biomembranes and, in particular, to a method for extracting an exosome. The method for extracting an exosome includes the following steps: step 1. adding a whole blood sample onto a microfluidic centrifuge disk; step 2. separating the whole blood to obtain plasma; step 3. filtering the plasma with a microfiltration membrane; step 4. mixing the filtered plasma with a loading solution to obtain a mixed solution; step 5. filtering the mixed solution with a surface-modified membrane to trap the exosome on the surface-modified membrane; and step 6. eluting the exosome from the surface-modified membrane to obtain a target exosome.

### CROSS-REFERENCE TO RELATED APPLICATION

This application is a continuation of PCT application serial no. PCT/CN2022/082583, filed on Mar. 23, 2022, which claims the priority and benefit of Chinese patent application serial no. 202111602970.5, filed on Dec. 24, 2021. The entireties of PCT application serial no. PCT/CN2022/082583 and Chinese patent application serial no. 202111602970.5 are hereby incorporated by reference herein and made a part of this specification.

### TECHNICAL FIELD

The present application relates to the technical field of biomembranes and, more particularly, to a method for extracting an exosome.

### BACKGROUND ART

Filter membranes are commonly used material in the biological field. Depending on different functional needs, different membranes will be used. For the use of membranes, they are generally used for the isolation and extraction of functional substances, such as the isolation and/or extraction of proteins, cells, extracellular secretory membrane structures (vesicles, etc.). For the extracellular secretory membrane structures, such as vesicles and exosomes, there are a large number of important information proteins such as transmembrane proteins and floating proteins on their membranes; and inside the membrane structures, there are nucleic acids, programmed cell death-related proteins, etc. Therefore, important information is carried within the extracellular secretory membrane structures. Many extracellular secretory membrane structures participate in the transfer of metabolic waste and also play an important role in pathophysiological activities.

Methods for obtaining extracellular secretory membrane structures include ultracentrifugation method, filtration centrifugation method, immunomagnetic bead method, PEG precipitation method, and kits. Among them, the ultracentrifugation method is the most commonly used one, with time-consuming extraction process, complex procedures, low yield of target extracts and low isolation efficiency. The filtration centrifugation method uses a filtration membrane to centrifuge and isolate an extracellular secretory membrane structure, and a target extract obtained by this method has the problem of low purity. The immunomagnetic bead method can obtain a target protein content twice higher than that of the ultracentrifugation method and the filtration centrifugation method; however, it cannot be applied to the extraction of all extracellular secretory membrane structures and is inefficient and expensive. An extracellular secretory membrane structure obtained by the PEG precipitation method has low purity and recovery rate and uneven particle size, and contains many impurity proteins, with production of polymers that are difficult to remove. Currently, commercially available exosome extraction kits are mainly used for extracellular vesicle extraction based on the PEG precipitation method. The most commonly used kits are ExoQuick, miRCURY, TEIR, etc. The kit-based methods are easy to operate, but the extracellular vesicles obtained contain many impurities. To sum up, there is still no absolute method or kit that can isolate ideal extracellular vesicles from various samples, with all requirements being met. Existing biomembranes are also difficult to meet the requirements for efficient and low-cost isolation and extraction.

### SUMMARY

In view of the problems of high cost, low efficiency and complexity in the existing extraction process for extracellular secretory membrane structures, the present application mainly provides a surface-modified membrane and a use thereof. The surface-modified membrane has the advantages of high efficiency and low cost for extracting an extracellular secretory membrane structure.

In a first aspect, the present application provides a surface-modified membrane and adopts the following technical solution:

A surface-modified membrane, including a base membrane and a surfactant loaded on the base membrane, wherein the surfactant includes at least one of a cationic surfactant and an amphoteric surfactant.

Optionally, the cationic surfactant includes one or more of stearamidopropyl dimethylamine, fatty acid potassium soap, sodium cocoyl glutamate, behenamidopropyl dimethylamine, quaternary ammonium salt Gemini surfactant, dodecyl trimethyl ammonium bromide, cetyl trimethyl ammonium bromide, polyoxyethylene trimethyl ammonium chloride, N-tetradecyl-N-(2-hydroxyethyl)-N,N-dimethyl ammonium bromide, N-tetradecyl-N,N-di(2-hydroxyethyl)-N-methyl ammonium bromide, QAS_{Cn}, polyalkyl trialkyl ammonium chloride, 3-chloro-2-hydroxypropyl trimethyl ammonium oxide, N-hexadecyl-N-(2-hydroxyethyl)-N,N-dimethyl ammonium bromide, N-hexadecyl-N,N-di(2-hydroxyethyl)-N-methyl ammonium bromide and 2,4,4-trichloro-2-dihydroxydiphenyl ether.

Optionally, the amphoteric surfactant is a betaine-type amphoteric surfactant, and the betaine-type amphoteric surfactant includes at least one of cocamidopropyl betaine, dodecyl betaine, octyldecylamidopropyl betaine and lauramidopropyl hydroxysultaine.

Optionally, the surfactant includes a cationic surfactant and an amphoteric surfactant, and a molar ratio of the cationic surfactant to the amphoteric surfactant is (0.01-1.64):3.

Optionally, the surface-modified membrane is also loaded with a protective agent, and the protective agent includes at least one of an alkaloid, an amino acid and a bioalcohol.

Optionally, a molar ratio of the alkaloid to the surfactant is 0.8-2.3:1.

Optionally, a molar ratio of the amino acid to the surfactant is 0.05-1.2:1.

Optionally, a molar ratio of the bioalcohol to the surfactant is 0.24-1.5:1.

In a second aspect, the present application provides a use of a surface-modified membrane and adopts the following technical solution:

A use of a surface-modified membrane, wherein the use includes use in isolation and/or extraction of an extracellular secretory membrane structure.

Optionally, the extracellular secretory membrane structure includes an extracellular vesicle.

In a third aspect, the present application provides a method for extracting an extracellular secretory membrane structure and adopts the following technical solution:

A method for extracting an extracellular secretory membrane structure, including the following steps:
S1. pretreating a biological sample to remove large particles;
S2. mixing the pretreated biological sample with a loading solution, and then centrifuging the resulting mixed solution and allowing the mixed solution to pass through the surface-modified membrane described above, so that an extracellular secretory membrane structure in the biological sample is captured on the surface-modified membrane, wherein the surface-modified membrane is loaded with a surfactant, and the surfactant includes at least one of a quaternary ammonium salt cationic surfactant and an amphoteric surfactant; and
S3. eluting the surface-modified membrane with an eluent to obtain a collection fluid containing the extracellular secretory membrane structure.

Optionally, the cationic surfactant includes one or more of stearamidopropyl dimethylamine, fatty acid potassium soap, sodium cocoyl glutamate, behenamidopropyl dimethylamine, quaternary ammonium salt Gemini surfactant, dodecyl trimethyl ammonium bromide, cetyl trimethyl ammonium bromide, polyoxyethylene trimethyl ammonium chloride, N-tetradecyl-N-(2-hydroxyethyl)-N,N-dimethyl ammonium bromide, N-tetradecyl-N,N-di(2-hydroxyethyl)-N-metyhl ammonium bromide, QAS_{Cn}, polyalkyl trialkyl ammonium chloride, 3-chloro-2-hydroxypropyl trimethyl ammonium oxide, N-hexadecyl-N-(2-hydroxyethyl)-N,N-dimethyl ammonium bromide, N-hexadecyl-N,N-di(2-hydroxyethyl)-N-methyl ammonium bromide and 2,4,4-trichloro-2-dihydroxydiphenyl ether, where n is a natural number less than or equal to 18.

Optionally, the quaternary ammonium salt Gemini surfactant is a symmetric Gemini surfactant or an asymmetric Gemini surfactant.

Optionally, the amphoteric surfactant is a betaine-type amphoteric surfactant, and the betaine-type amphoteric surfactant includes at least one of cocamidopropyl betaine, dodecyl betaine, octyldecylamidopropyl betaine and lauramidopropyl hydroxysultaine.

Optionally, the surfactant includes a quaternary ammonium salt cationic surfactant and an amphoteric surfactant.

Optionally, a molar ratio of the quaternary ammonium salt cationic surfactant to the amphoteric surfactant is (0.01-1.64):3.

Optionally, in a case where the quaternary ammonium salt cationic surfactant includes one or more of dodecyl trimethyl ammonium bromide (DTAB), cetyl trimethyl ammonium bromide (CTAB), polyoxyethylene trimethyl ammonium chloride, N-tetradecyl-N-(2-hydroxyethyl)-N,N-dimethyl ammonium bromide, N-tetradecyl-N,N-di(2-hydroxyethyl)-N-methyl ammonium bromide, polyalkyl trialkyl ammonium chloride, 3-chloro-2-hydroxypropyl trimethyl ammonium oxide, N-hexadecyl-N-(2-hydroxyethyl)-N,N-dimethyl ammonium bromide, N-hexadecyl-N,N-di (2-hydroxyethyl)-N-methyl ammonium bromide and 2,4,4-trichloro-2-dihydroxydiphenyl ether, the molar ratio of the quaternary ammonium salt cationic surfactant to the amphoteric surfactant is (0.01-0.36):3.

Optionally, in a case where the quaternary ammonium salt cationic surfactant is a symmetric Gemini surfactant, the molar ratio of the quaternary ammonium salt cationic surfactant to the amphoteric surfactant is (0.23-0.45):3.

Optionally, in a case where the quaternary ammonium salt cationic surfactant is an asymmetric Gemini surfactant, the molar ratio of the quaternary ammonium salt cationic surfactant to the amphoteric surfactant is (0.56-1.26):3.

Optionally, during preparation of the surface-modified membrane, a protective agent is also added together with the surfactant; the protective agent includes at least one of an alkaloid, an amino acid and a bioalcohol.

Optionally, the alkaloid is a betaine.

Optionally, a molar ratio of the alkaloid to the surfactant is 0.8-2.3:1.

Optionally, the amino acid is proline.

Optionally, a molar ratio of the amino acid to the surfactant is 0.05-1.2:1.

Optionally, the bioalcohol is mannitol.

Optionally, a molar ratio of the bioalcohol to the surfactant is 0.24-1.5:1.

By loading the protective agent on the surface-modified membrane, the possibility of the extracellular secretory membrane structure being damaged in a salt environment can be reduced. The alkaloid, especially the betaine, can resist salt stress and thus protect the extracellular secretory membrane structure. The bioalcohol, especially the mannitol, also protects the extracellular secretory membrane structure from rupture to a certain extent. The amino acid, especially the proline, protects the extracellular secretory membrane structure by means of scavenging free radicals or the like.

In a case where the alkaloid, the amino acid and the bioalcohol are used in combination, a better protective effect on the extracellular secretory membrane structure can be achieved.

Optionally, the loading solution includes the following components of certain concentrations:

87-113 mM tris-propane buffer and 130-165 mM NaCl, pH 6.23-6.78.

Optionally, the loading solution further includes a protective agent; the protective agent includes at least one of an alkaloid, an amino acid and a bioalcohol.

By adding the protective agent to the loading solution, the extracellular secretory membrane structure is protected from being easily damaged in a salt environment.

Optionally, if the loading solution contains the alkaloid, a molar ratio of the alkaloid to the NaCl is 0.25-0.46:1.

Optionally, if the loading solution contains the amino acid, a molar ratio of the amino acid to the NaCl is 0.13-0.35:1.

Optionally, if the loading solution contains the bioalcohol, a molar ratio of the bioalcohol to the NaCl is 0.12-0.19:1.

Optionally, the eluent includes the following components of certain concentrations:

38-62 mM tris-propane buffer and 0.8-2.3 M NaCl, pH 6.13-6.52.

Optionally, the loading solution further includes a protective agent; the protective agent includes at least one of an alkaloid, an amino acid and a bioalcohol.

Optionally, if the loading solution contains the alkaloid, a molar ratio of the alkaloid to the NaCl is 0.02-0.16:1.

Optionally, if the loading solution contains the amino acid, a molar ratio of the amino acid to the NaCl is 0.005-0.1:1.

Optionally, if the loading solution contains the bioalcohol, a molar ratio of the bioalcohol to the NaCl is 0.005-0.1:1.

In a fourth aspect, the present application provides a kit for implementing the extraction method described above, which adopts the following technical solution:

A kit for implementing the extraction method described above, including the loading solution, the eluent and a filtering device, wherein the filtering device contains the surface-modified membrane.

To sum up, the present application has the following beneficial effects:
1. In the present application, the surface-modified membrane loaded with an appropriate surfactant is used. The surfactant includes at least one of a cationic surfactant and an amphoteric surfactant to achieve efficient isolation and extraction of an extracellular secretory membrane structure in a biological sample.
2. In the present application, the protective agent is added to the surface-modified membrane to further prevent the extracellular secretory membrane structure from being damaged, thereby ensuring the integrity of the obtained extracellular secretory membrane structure and further increasing the concentration of the obtained extracellular secretory membrane structure.

### BRIEF DESCRIPTION OF THE DRAWINGS

FIG. 1 is an overall schematic structural diagram of an isolation and extraction device according to an embodiment of the present application;

FIG. 2 is an overall schematic structural diagram of an isolation and extraction device according to another embodiment of the present application;

FIG. 3 is an overall schematic structural diagram of an isolation and extraction device according to yet another embodiment of the present application;

FIG. 4 is a top view of a bottom plate according to yet another embodiment of the present application;

FIG. 5 is a schematic diagram showing the structure of a sample injection unit according to yet another embodiment of the present application;

FIG. 6 is a schematic diagram showing the relevant structure of a sample quantification pool according to yet another embodiment of the present application;

FIG. 7 is a schematic diagram showing the structure of a sample pretreatment unit according to yet another embodiment of the present application;

FIG. 8 is a schematic diagram showing the structure of an impurity filter pool according to yet another embodiment of the present application;

FIG. 9 is a schematic diagram showing the structure of a loading solution container assembly according to yet another embodiment of the present application;

FIG. 10 is a schematic diagram showing the internal structure of a loading solution container according to yet another embodiment of the present application;

FIG. 11 is a schematic diagram showing the structure of an eluent pool and a carrier filter pool according to yet another embodiment of the present application;

FIG. 12 is a schematic diagram showing the internal structure of the carrier filter pool according to yet another embodiment of the present application;

FIG. 13 is a schematic diagram showing the structure of an eluent container assembly according to yet another embodiment of the present application;

FIG. 14 is a schematic diagram showing the internal structure of the eluent container assembly according to yet another embodiment of the present application;

FIG. 15 is a schematic diagram showing the structure of an enrichment unit according to yet another embodiment of the present application;

FIG. 16 is a schematic diagram showing distribution of weight-reduction balancing pools on the bottom plate according to yet another embodiment of the present application;

FIG. 17 is schematic structural diagram of another incubation pool of the present application;

FIG. 18 is a comparison chart of extraction time between different extraction methods used to extract exosomes, where, 1: microfluidic centrifugal disk extraction method of Example 10; 2: size exclusion chromatography of Comparative Example 1; 3: ultracentrifugation method of Comparative Example 2; 4: PEG precipitation method of Comparative Example 3; 5: density gradient centrifugation method of Comparative Example 4;

FIG. 19 is a comparison chart of Western Blot results of exosomes extracted from different samples using the method of Example 10;

FIG. 20 is a comparison chart of Western Blot results of exosomes in cell supernatants obtained when the exosomes were extracted using different extraction methods, where \textcircled{1}: microfluidic centrifugal disk extraction method of Example 10; \textcircled{2}: size exclusion chromatography of Comparative Example 1; \textcircled{3}: ultracentrifugation method of Comparative Example 2; \textcircled{4}: PEG precipitation method of Comparative Example 3; \textcircled{5}: density gradient centrifugation method of Comparative Example 4;

FIG. 21 is a TEM image of exosomes extracted using the method of Example 10;

FIG. 22 is a SEM image of exosomes extracted using the method of Example 10; and

FIG. 23 is an NTA diagram of exosomes extracted using the method of Example 10.

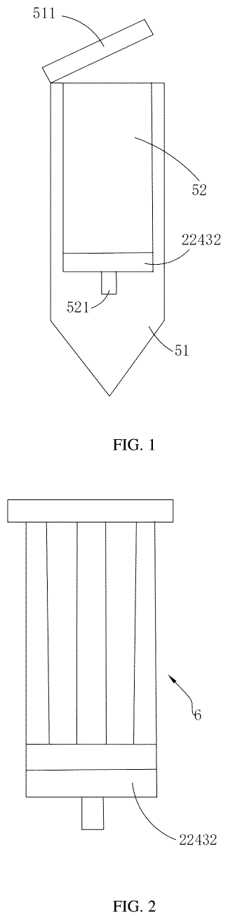

### DETAILED DESCRIPTION

The present application provides a surface-modified membrane, including a base membrane and a surfactant loaded on the base membrane, wherein the surfactant includes at least one of a cationic surfactant and an amphoteric surfactant.

In some embodiments, the cationic surfactant includes one or more of stearamidopropyl dimethylamine, fatty acid potassium soap, sodium cocoyl glutamate, behenamidopropyl dimethylamine, quaternary ammonium salt Gemini surfactant, dodecyl trimethyl ammonium bromide, cetyl trimethyl ammonium bromide, polyoxyethylene trimethyl ammonium chloride, N-tetradecyl-N-(2-hydroxyethyl)-N,N-dimethyl ammonium bromide, N-tetradecyl-N,N-di(2-hydroxyethyl)-N-methyl ammonium bromide, QAS_{Cn}, polyalkyl trialkyl ammonium chloride, 3-chloro-2-hydroxypropyl trimethyl ammonium oxide, N-hexadecyl-N-(2-hydroxyethyl)-N,N-dimethyl ammonium bromide, N-hexadecyl-N,N-di(2-hydroxyethyl)-N-methyl ammonium bromide and 2,4,4-trichloro-2-dihydroxydiphenyl ether.

In some embodiments, the amphoteric surfactant is a betaine-type amphoteric surfactant, and the betaine-type amphoteric surfactant includes at least one of cocamidopropyl betaine, dodecyl betaine, octyldecylamidopropyl betaine and lauramidopropyl hydroxysultaine.

In some embodiments, the cationic surfactant is a quaternary ammonium salt cationic surfactant, and the quaternary ammonium salt cationic surfactant includes one or more of stearamidopropyl dimethylamine, fatty acid potassium soap, sodium cocoyl glutamate, behenamidopropyl dimethylamine, quaternary ammonium salt Gemini surfactant, dodecyl trimethyl ammonium bromide, cetyl trimethyl ammonium bromide, polyoxyethylene trimethyl ammonium chloride, N-tetradecyl-N-(2-hydroxyethyl)-N,N-dimethyl ammonium bromide, N-tetradecyl-N,N-di(2-hydroxyethyl)-N-methyl ammonium bromide, QAS_{Cn}, polyalkyl trialkyl ammonium chloride, 3-chloro-2-hydroxypropyl trimethyl ammonium oxide, N-hexadecyl-N-(2-hydroxyethyl)-N,N-dimethyl ammonium bromide, N-hexadecyl-N,N-di(2-hydroxyethyl)-N-methyl ammonium bromide and 2,4,4-trichloro-2-dihydroxydiphenyl ether.

In some embodiments, the quaternary ammonium salt cationic surfactant is a quaternary ammonium salt Gemini surfactant.

In some embodiments, the quaternary ammonium salt Gemini surfactant is a symmetric Gemini surfactant or an asymmetric Gemini surfactant.

The quaternary ammonium salt Gemini surfactant is commercially available. The quaternary ammonium salt Gemini surfactant may be a quaternary ammonium salt Gemini surfactant purchased from Wuhan Huaxiang Kejie Biotechnology Co., Ltd or may be a quaternary ammonium salt Gemini surfactant purchased from Shanghai Shenrong Chemical Technology Co., Ltd.

In some embodiments, the quaternary ammonium salt Gemini surfactant is prepared.

In some embodiments, the synthesis of the asymmetric quaternary ammonium salt Gemini surfactant includes the following steps: allowing a short-chain alkyl tertiary amine to react with 1,3-dibromoisopropanol to obtain an intermediate product; and then allowing the intermediate product to react with a long-chain alkyl tertiary amine to obtain the quaternary ammonium salt Gemini surfactant, wherein an alkyl chain of the long-chain alkyl tertiary amine has 12, 16 or 18 carbon atoms, and an alkyl chain of the short-chain alkyl tertiary amine has 6 or 8 carbon atoms.

In some embodiments, the long-chain alkyl tertiary amine is any one of dodecyl tertiary amine, octadecyl tertiary amine and hexadecyl tertiary amine.

In some embodiments, the short-chain alkyl tertiary amine includes any one of octylamine and hexylamine.

In some embodiments, a molar ratio of 1,3-dibromoisopropanol to the short-chain alkyl tertiary amine is 3-5:1; further, the molar ratio of 1,3-dibromoisopropanol to the short-chain alkyl tertiary amine may be 3.5:1, 4:1 or 4.5:1. A molar ratio of the long-chain alkyl tertiary amine to the intermediate product is 0.8-1.2:1; further, the molar ratio of the long-chain alkyl tertiary amine to the intermediate product may be 0.9:1, 1:1 or 1.1:1.

In some embodiments, the alkyl chain of the long-chain alkyl tertiary amine has 18 carbon atoms, and the alkyl chain of the short chain alkyl tertiary amine has 6 carbon atoms.

In some embodiments, the symmetric quaternary ammonium salt Gemini surfactant is synthesized from a long-chain alkyl tertiary amine and a dibromoalkane or is synthesized from a long-chain alkyl tertiary amine, epichlorohydrin and hydrochloric acid; an alkyl chain of the long-chain alkyl tertiary amine has 8, 12 or 16 carbon atoms, and an alkyl chain of the dibromoalkane has 2, 3 or 4 carbon atoms.

In some embodiments, the alkyl chain of the long-chain alkyl tertiary amine has 12 or 16 carbon atoms.

In some embodiments, the long-chain alkyl tertiary amine is any one of N,N-dimethyldodecylamine, N,N-dimethyl-1,3-propylenediamine and N,N-dimethylhexadecyl-amine; the dibromoalkane is 1,3-dibromopropane.

In some embodiments, the quaternary ammonium salt cationic surfactant is QAS_{Cn}, where n is any one of 0, 6, 8, 12, 14, and 18.

In some embodiments, the amphoteric surfactant is a betaine-type amphoteric surfactant. The betaine-type amphoteric surfactant may be cocamidopropyl betaine, e.g., a product CAB-35, or may be dodecyl betaine, e.g., a product BS-12, or may be octyldecylamidopropyl betaine. In some embodiments, the betaine-type amphoteric surfactant is a sulfobetaine amphoteric surfactant. The betaine-type amphoteric surfactant may further be 3-(N,N-dimethyldodecyl ammonium) propane sulfonate (SPPT), or may also be lauramidopropyl hydroxysultaine, e.g., a product LHSB-35.

In some embodiments, the base membrane is an RC membrane.

In some embodiments, the surface-modified membrane is prepared by a method including the following steps: completely dissolving at least one of the quaternary ammonium salt cationic surfactant and the amphoteric surfactant in a first organic solvent; then, placing the base membrane in the resulting mixed solution, and allowing reaction with shaking at room temperature and then drying the base membrane; and then, washing the base membrane with a second organic solvent and then drying the base membrane, thus obtaining the surface-modified membrane.

In some embodiments, a time of shaking at room temperature is within a range of 40 h to 56 h. In some embodiments, the time of shaking at room temperature may be 43 h, 46 h, 49 h, 52 h, or 54 h.

In some embodiments, the first organic solvent is methanol.

In some embodiments, the second organic solvent is an ethanol solution, and the ethanol solution is a 5-95% ethanol solution. In a case where the second organic solvent is used for washing the base membrane, the base membrane may be washed once with 95% ethanol; alternatively, the base membrane may be gradient washed with ethanol solutions of different concentrations in a decreasing order.

In some embodiments, the surfactant loaded during the preparation of the surface-modified membrane is a quaternary ammonium salt cationic surfactant or an amphoteric surfactant.

In some embodiments, during the preparation of the surface-modified membrane, the surfactant includes a quaternary ammonium salt cationic surfactant and an amphoteric surfactant. Optionally, a molar ratio of the quaternary ammonium salt cationic surfactant to the amphoteric surfactant is (0.01-1.64):3.

In some embodiments, during the preparation of the surface-modified membrane, the surfactant includes a quaternary ammonium salt cationic surfactant and an amphoteric surfactant; and in a case where the quaternary ammonium salt cationic surfactant includes one or more of dodecyl trimethyl ammonium bromide (DTAB), cetyl trimethyl ammonium bromide (CTAB), polyoxyethylene trimethyl ammonium chloride, N-tetradecyl-N-(2-hydroxyethyl)-N,N-dimethyl ammonium bromide, N-tetradecyl-N,N-di(2-hydroxyethyl)-N-methyl ammonium bromide, polyalkyl trialkyl ammonium chloride, 3-chloro-2-hydroxypropyl trimethyl ammonium oxide, N-hexadecyl-N-(2-hydroxyethyl)-N,N-dimethyl ammonium bromide, N-hexadecyl-N,N-di(2-hydroxyethyl)-N-methyl ammonium bromide and 2,4,4-trichloro-2-dihydroxydiphenyl ether, the molar ratio of the quaternary ammonium salt cationic surfactant to the amphoteric surfactant is (0.01-0.36):3. The molar ratio may be, for example, 0.05:3, 0.1:3, 0.15:3, 0.2:3, 0.25:3, or 0.3:3. In some embodiments, during the preparation of the surface-modified membrane, the surfactant includes a quaternary ammonium salt cationic surfactant and an amphoteric surfactant; and in a case where the quaternary ammonium salt cationic surfactant is a symmetric Gemini surfactant, a molar ratio of the quaternary ammonium salt cationic surfactant to the amphoteric surfactant is (0.23-0.45):3. The molar ratio may be, for example, 0.25:3, 0.31:3, 0.38:3, or 0.42:3. In some embodiments, during the preparation of the surface-modified membrane, the surfactant includes a quaternary ammonium salt cationic surfactant and an amphoteric surfactant; and in a case where the quaternary ammonium salt cationic surfactant is an asymmetric Gemini surfactant, a molar ratio of the quaternary ammonium salt cationic surfactant to the amphoteric surfactant is (0.56-1.26):3. The molar ratio may be, for example, 0.65:3, 0.72:3, 0.84:3, 0.92:3, 1.06:3, or 1.18:3. In some embodiments, during the preparation of the surface-modified membrane, the surfactant includes a quaternary ammonium salt cationic surfactant and an amphoteric surfactant; and in a case where the quaternary ammonium salt cationic surfactant is QAS_{Cn} (n is 0 or 18) and the amphoteric surfactant is 3-(N,N-dimethyldodecyl ammonium) propane sulfonate, a molar ratio of the quaternary ammonium salt cationic surfactant to the amphoteric surfactant is (1.05-1.64):3. The molar ratio may be, for example, 1.16:3, 1.28:3, 1.36:3, or 1.52:3.

In some embodiments, during the preparation of the surface-modified membrane, a protective agent is also added together with the surfactant; the protective agent is added to prevent the exosome from being damaged by osmotic pressure in a high-salt environment. The protective agent includes at least one of an alkaloid, an amino acid and a bioalcohol. In some embodiments, the protective agent includes an alkaloid and an amino acid. In some embodiments, the protective agent includes an alkaloid, a bioalcohol, and an amino acid.

In some embodiments, the alkaloid is a betaine. In some embodiments, a molar ratio of the alkaloid to the surfactant is 0.8-2.3:1. Optionally, the molar ratio of the alkaloid to the surfactant is 1.3:1, 1.6:1, 1.8:1, or 2.2:1.

In some embodiments, the amino acid is proline. In some embodiments, a molar ratio of the amino acid to the surfactant is 0.05-1.2:1. Optionally, the molar ratio of the amino acid to the surfactant is 0.1:1, 0.3:1, 0.7:1, 0.9:1, or 1.1:1.

In some embodiments, the bioalcohol is mannitol. In some embodiments, a molar ratio

of the bioalcohol to the surfactant is 0.24-1.5:1. Optionally, the molar ratio of the bioalcohol to the surfactant is 0.3:1, 0.46:1, 0.69:1, 0.82:1, 0.96:1, 1.14:1, 1.23:1, or 1.41:1.

The present application further provides a use of the surface-modified membrane, wherein the use includes use in isolation and/or extraction of an extracellular secretory membrane structure.

The term “use in isolation and/or extraction of an extracellular secretory membrane structure”, as used herein, means only use in isolation of an extracellular secretory membrane structure, or only use in extraction of an extracellular secretory membrane structure, or use in isolation and extraction of an extracellular secretory membrane structure.

In some embodiments, the extracellular secretory membrane structure includes an extracellular vesicle and an exosome.

The present application further provides a method for extracting an extracellular secretory membrane structure, including the following steps:
S1. pretreating a biological sample;
S2. mixing the pretreated biological sample with a loading solution, and then centrifuging the resulting mixed solution and allowing the mixed solution to pass through a surface-modified membrane, so that an extracellular secretory membrane structure in the biological sample is captured on the surface-modified membrane, wherein the surface-modified membrane is loaded with a surfactant, and the surfactant includes at least one of a cationic surfactant and an amphoteric surfactant; and
S3. eluting the surface-modified membrane with an eluent to obtain a collection fluid containing the extracellular secretory membrane structure.

The biological sample that may be treated by the method of the present application includes, but is not limited to, whole blood, plasma, serum, urine, saliva, hydrothorax and ascites, cerebrospinal fluid, cell culture supernatant, tears, semen, amniotic fluid, gastric juice, saliva, nasal discharge, bronchoalveolar lavage fluid, joint synovial fluid, bile, uterine mucus and feces.

The extracellular secretory membrane structure includes an extracellular vesicle and an exosome. The exosome refers to an extracellular vesicle containing complex RNAs and proteins, with a size of about 30 nm to 150 nm. They are mainly derived from multivesicular bodies formed by the invagination of intracellular lysosomal particles, and are released into the extracellular matrix after the fusion of the outer membrane of multivesicular bodies and the cell membrane. Exosomes are currently viewed as specifically secreted membrane vesicles that participate in intercellular communication, carry rich molecular information on original cells, and are considered potential markers for early tumor diagnosis. Therefore, it is crucial to develop rapid and easy extraction methods for tumor exosomes.

At present, the extraction of exosomes is mainly implemented by ultracentrifugation method, filtration centrifugation method, immunomagnetic bead method, PEG precipitation method and kits. Among them, the ultracentrifugation method is the most commonly used one for extracting exosomes, with time-consuming extraction process, complex procedures, low exosome yield and low isolation efficiency. The filtration centrifugation method uses a filtration membrane to centrifuge and isolate exosomes, and the exosomes obtained by this method have the problem of low purity. The protein content of exosomes obtained by the immunomagnetic bead method is twice higher than that of exosomes obtained by the ultracentrifugation method and the filtration centrifugation method; however, it cannot be applied to the extraction of all exosomes and is inefficient and expensive. The exosome obtained by the PEG precipitation method has low purity, low recovery rate and uneven particle size and contains many impurity proteins, with production of polymers that are difficult to remove. Currently, commercially available exosome extraction kit methods are mainly used for exosome extraction based on the PEG precipitation method. The most commonly used kits are ExoQuick, miRCURY, TEIR, etc. The kit-based methods are easy to operate, but the exosomes obtained contain many impurities. To sum up, there is still no absolute method or kit that can isolate ideal exosomes from various samples, with all requirements being met.

The pretreatment step described in step S1 is to remove cell impurities, cell debris, dead corpuscles, large-diameter vesicles and other large particles out of the biological sample. The pretreatment step may be to filter the biological sample by a microfiltration membrane. The microfiltration membrane that can be used here may be a 0.22 μm filter membrane for removing impurities with a particle size greater than about 200 nm; Specifically, the microfiltration membrane may be, but is not limited to, a polyethersulfone (PES) hydrophilic filter membrane purchased from Beijing Mreda Technology Co., Ltd.

In some embodiments, step S1 further includes a centrifugation step after membrane filtering treatment and the centrifugation is performed at a speed of 2500-3500 rpm for 150-250 s.

The pretreated biological sample is then mixed with the loading solution and then passes through the surface-modified membrane, and finally the surface-modified membrane is eluted with an eluent. In this treatment step, the loading solution, the surface-modified membrane and the eluent are used in combination: the loading solution is used to surface modify the extracellular secretory membrane structure in the biological sample, so that the extracellular secretory membrane structure is attached to the surface-modified membrane, thus facilitating the acquisition of an exosome with high purity and high recovery rate. After the biological sample mixed with the loading solution passes through the surface-modified membrane, the extracellular secretory membrane structure in the biological sample can bind well to the quaternary ammonium salt cationic surfactant and/or the amphoteric surfactant on the surface-modified membrane due to its negatively charged surface, thereby achieving the isolation of the extracellular secretory membrane structure from other substances in the biological sample. Then, an elution step is performed, where the selected eluent has a stronger binding force with the exosome than the cationic surfactant and/or the amphoteric surfactant on the surface-modified membrane, so that the extracellular secretory membrane structure is eluted from the surface-modified membrane, thereby obtaining the collection fluid containing the extracellular secretory membrane structure. Centrifugation is then performed to obtain the extracellular secretory membrane structure.

In some optional embodiments, the cationic surfactant includes one or more of stearamidopropyl dimethylamine, fatty acid potassium soap, sodium cocoyl glutamate, behenamidopropyl dimethylamine, quaternary ammonium salt Gemini surfactant, dodecyl trimethyl ammonium bromide (DTAB), cetyl trimethyl ammonium bromide (CTAB), polyoxyethylene trimethyl ammonium chloride, N-tetradecyl-N-(2-hydroxyethyl)-N,N-dimethyl ammonium bromide, N-tetradecyl-N,N-di(2-hydroxyethyl)-N-methyl ammonium bromide, QAS_{Cn}, polyalkyl trialkyl ammonium chloride, 3-chloro-2-hydroxypropyl trimethyl ammonium oxide, N-hexadecyl-N-(2-hydroxyethyl)-N,N-dimethyl ammonium bromide, N-hexadecyl-N,N-di(2-hydroxyethyl)-N-methyl ammonium bromide and 2,4,4-trichloro-2-dihydroxydiphenyl ether, where n is a natural number less than or equal to 18.

In some embodiments, the quaternary ammonium salt cationic surfactant is a quaternary ammonium salt Gemini surfactant.

In some embodiments, the quaternary ammonium salt Gemini surfactant is a symmetric Gemini surfactant or an asymmetric Gemini surfactant.

The quaternary ammonium salt Gemini surfactant is commercially available. The quaternary ammonium salt Gemini surfactant may be a quaternary ammonium salt Gemini surfactant purchased from Wuhan Huaxiang Kejie Biotechnology Co., Ltd or may be a quaternary ammonium salt Gemini surfactant purchased from Shanghai Shenrong Chemical Technology Co., Ltd.

In some embodiments, the quaternary ammonium salt Gemini surfactant is prepared.

In some embodiments, the synthesis of the asymmetric quaternary ammonium salt Gemini surfactant includes the following steps: allowing a short-chain alkyl tertiary amine to react with 1,3-dibromoisopropanol to obtain an intermediate product; and then allowing the intermediate product to react with a long-chain alkyl tertiary amine to obtain the quaternary ammonium salt Gemini surfactant, wherein an alkyl chain of the long-chain alkyl tertiary amine has 12, 16 or 18 carbon atoms, and an alkyl chain of the short-chain alkyl tertiary amine has 6 or 8 carbon atoms.

In some embodiments, the long-chain alkyl tertiary amine is any one of dodecyl tertiary amine, octadecyl tertiary amine and hexadecyl tertiary amine.

In some embodiments, the short-chain alkyl tertiary amine includes any one of octylamine and hexylamine.

In some embodiments, a molar ratio of 1,3-dibromoisopropanol to the short-chain alkyl tertiary amine is 3-5:1; further, the molar ratio of 1,3-dibromoisopropanol to the short-chain alkyl tertiary amine may be 3.5:1, 4:1 or 4.5:1. A molar ratio of the long-chain alkyl tertiary amine to the intermediate product is 0.8-1.2:1; further, the molar ratio of the long-chain alkyl tertiary amine to the intermediate product may be 0.9:1, 1:1 or 1.1:1.

In some embodiments, the alkyl chain of the long-chain alkyl tertiary amine has 18 carbon atoms, and the alkyl chain of the short chain alkyl tertiary amine has 6 carbon atoms.

In some embodiments, the symmetric quaternary ammonium salt Gemini surfactant is synthesized from a long-chain alkyl tertiary amine and a dibromoalkane or is synthesized from a long-chain alkyl tertiary amine, epichlorohydrin and hydrochloric acid; an alkyl chain of the long-chain alkyl tertiary amine has 8, 12 or 16 carbon atoms, and an alkyl chain of the dibromoalkane has 2, 3 or 4 carbon atoms.

In some embodiments, the alkyl chain of the long-chain alkyl tertiary amine has 12 or 16 carbon atoms.

In some embodiments, the long-chain alkyl tertiary amine is any one of N,N-dimethyldodecylamine, N,N-dimethyl-1,3-propylenediamine and N,N-dimethylhexadecyl-amine; the dibromoalkane is 1,3-dibromopropane.

In some embodiments, the quaternary ammonium salt cationic surfactant is QAS_{Cn}, where n is any one of 0, 6, 8, 12, 14, and 18.

In some embodiments, n is 0 or 18.

In some embodiments, the amphoteric surfactant is a betaine-type amphoteric surfactant. The betaine-type amphoteric surfactant may be cocamidopropyl betaine, e.g., a product CAB-35, or may be dodecyl betaine, e.g., a product BS-12, or may be octyldecylamidopropyl betaine. In some embodiments, the betaine-type amphoteric surfactant is a sulfobetaine amphoteric surfactant. The betaine-type amphoteric surfactant may further be 3-(N,N-dimethyldodecyl ammonium) propane sulfonate (SPPT), or may also be lauramidopropyl hydroxysultaine, e.g., a product LHSB-35.

In some embodiments, the surface-modified membrane is prepared by a method including the following steps: completely dissolving at least one of the quaternary ammonium salt cationic surfactant and the amphoteric surfactant in a first organic solvent; then, placing the base membrane in the resulting mixed solution, and allowing reaction with shaking at room temperature and then drying the base membrane; and then, washing the base membrane with a second organic solvent and then drying the base membrane, thus obtaining the surface-modified membrane.

In some embodiments, the base membrane is an RC membrane.

In some embodiments, a time of shaking at room temperature is within a range of 40 h to 56 h. In some embodiments, the time of shaking at room temperature may be 43 h, 46 h, 49 h, 52 h, or 54 h.

In some embodiments, the first organic solvent is methanol.

In some embodiments, the second organic solvent is an ethanol solution, and the ethanol solution is a 5-95% ethanol solution. In a case where the second organic solvent is used for washing the base membrane, the base membrane may be washed once with 95% ethanol; alternatively, the base membrane may be gradient washed with ethanol solutions of different concentrations in a decreasing order.

In some embodiments, the surfactant loaded during the preparation of the surface-modified membrane is a quaternary ammonium salt cationic surfactant or an amphoteric surfactant.

In some embodiments, during the preparation of the surface-modified membrane, the surfactant includes a quaternary ammonium salt cationic surfactant and an amphoteric surfactant. Optionally, a molar ratio of the quaternary ammonium salt cationic surfactant to the amphoteric surfactant is (0.01-1.64):3.

In some embodiments, during the preparation of the surface-modified membrane, the surfactant includes a quaternary ammonium salt cationic surfactant and an amphoteric surfactant; and in a case where the quaternary ammonium salt cationic surfactant includes one or more of dodecyl trimethyl ammonium bromide (DTAB), cetyl trimethyl ammonium bromide (CTAB), polyoxyethylene trimethyl ammonium chloride, N-tetradecyl-N-(2-hydroxyethyl)-N,N-dimethyl ammonium bromide, N-tetradecyl-N,N-di(2-hydroxyethyl)-N-methyl ammonium bromide, polyalkyl trialkyl ammonium chloride, 3-chloro-2-hydroxypropyl trimethyl ammonium oxide, N-hexadecyl-N-(2-hydroxyethyl)-N,N-dimethyl ammonium bromide, N-hexadecyl-N,N-di(2-hydroxyethyl)-N-methyl ammonium bromide and 2,4,4-trichloro-2-dihydroxydiphenyl ether, the molar ratio of the quaternary ammonium salt cationic surfactant to the amphoteric surfactant is (0.01-0.36):3. The molar ratio may be, for example, 0.05:3, 0.1:3, 0.15:3, 0.2:3, 0.25:3, or 0.3:3. In some embodiments, during the preparation of the surface-modified membrane, the surfactant includes a quaternary ammonium salt cationic surfactant and an amphoteric surfactant; and in a case where the quaternary ammonium salt cationic surfactant is a symmetric Gemini surfactant, a molar ratio of the quaternary ammonium salt cationic surfactant to the amphoteric surfactant is (0.23-0.45):3. The molar ratio may be, for example, 0.25:3, 0.31:3, 0.38:3, or 0.42:3. In some embodiments, during the preparation of the surface-modified membrane, the surfactant includes a quaternary ammonium salt cationic surfactant and an amphoteric surfactant; and in a case where the quaternary ammonium salt cationic surfactant is an asymmetric Gemini surfactant, a molar ratio of the quaternary ammonium salt cationic surfactant to the amphoteric surfactant is (0.56-1.26) 3. The molar ratio may be, for example, 0.65:3, 0.72:3, 0.84:3, 0.92:3, 1.06:3, or 1.18:3. In some embodiments, during the preparation of the surface-modified membrane, the surfactant includes a quaternary ammonium salt cationic surfactant and an amphoteric surfactant; and in a case where the quaternary ammonium salt cationic surfactant is QAS_{Cn} (n is 0 or 18) and the amphoteric surfactant is 3-(N,N-dimethyldodecyl ammonium) propane sulfonate, a molar ratio of the quaternary ammonium salt cationic surfactant to the amphoteric surfactant is (1.05-1.64):3. The molar ratio may be, for example, 1.16:3, 1.28:3, 1.36:3, or 1.52:3.

In some embodiments, during the preparation of the surface-modified membrane, a protective agent is also added together with the surfactant; the protective agent is added to prevent the extracellular secretory membrane structure from being damaged by osmotic pressure in a high-salt environment. The protective agent includes at least one of an alkaloid, an amino acid and a bioalcohol. In some embodiments, the protective agent includes an alkaloid and an amino acid. In some embodiments, the protective agent includes an alkaloid, a bioalcohol, and an amino acid.

In some embodiments, the alkaloid is a betaine. In some embodiments, a molar ratio of the alkaloid to the surfactant is 0.8-2.3:1. Optionally, the molar ratio of the alkaloid to the surfactant is 1.3:1, 1.6:1, 1.8:1, or 2.2:1.

In some embodiments, the amino acid is proline. In some embodiments, a molar ratio of the amino acid to the surfactant is 0.05-1.2:1. Optionally, the molar ratio of the amino acid to the surfactant is 0.1:1, 0.3:1, 0.7:1, 0.9:1, or 1.1:1.

In some embodiments, the bioalcohol is mannitol. In some embodiments, a molar ratio of the bioalcohol to the surfactant is 0.24-1.5:1. Optionally, the molar ratio of the bioalcohol to the surfactant is 0.3:1, 0.46:1, 0.69:1, 0.82:1, 0.96:1, 1.14:1, 1.23:1, or 1.41:1.

In some embodiments, the loading solution includes NaCl with a concentration of 130-170 mM. Optionally, the concentration of the NaCl is 140 mM, 152 mM, 160 mM or 168 mM. In some embodiments, the loading solution further includes a tris-propane buffer with a concentration of 91-103 mM and pH of 6.45-6.7. Optionally, the concentration of the tris-propane buffer is 94 mM, 98 mM, or 102 mM.

In some embodiments, the loading solution further includes a protective agent, and the protective agent includes at least one of an alkaloid, an amino acid and a bioalcohol. In some embodiments, the protective agent includes an alkaloid and an amino acid. In some embodiments, the protective agent includes an alkaloid, a bioalcohol, and an amino acid.

In some embodiments, the alkaloid is a betaine. In some embodiments, a molar ratio of the alkaloid to the NaCl is 0.25-0.46:1. Optionally, the molar ratio of the alkaloid to the NaCl is 0.28:1, 0.32:1, 0.37:1, 0.4:1 or 0.43:1.

In some embodiments, the amino acid is proline. In some embodiments, a molar ratio of the amino acid to the NaCl is 0.13-0.35:1. Optionally, the molar ratio of the amino acid to the NaCl is 0.16:1, 0.19:1, 0.22:1, 0.26:1, 0.29:1, 0.32:1, or 0.34:1.

In some embodiments, the bioalcohol is mannitol. In some embodiments, a molar ratio of the bioalcohol to the NaCl is 0.12-0.19:1. Optionally, the molar ratio of the bioalcohol to the NaCl is 0.14:1, 0.16:1, or 0.18:1.

In some embodiments, a volume ratio of the biological sample to the loading solution is 0.4-1.6:1. Optionally, the volume ratio of the biological sample to the loading solution is 0.53:1, 0.68:1, 0.76:1, 0.94:1, 1.21:1, or 1.47:1.

In some embodiments, the eluent includes NaCl with a concentration of 1-2.5 M. Optionally, the concentration of the NaCl is 1.2 M, 1.45 M, 1.53 M, 1.62 M, 1.83 M, 1.96 M, or 2.37 M. In some embodiments, the eluent further includes a tris-propane buffer with a concentration of 42-58 mM and pH of 6.2-6.41. Optionally, the concentration of the tris-propane buffer is 45 mM, 48 mM, 53 mM, or 56 mM.

In some embodiments, the eluent further includes the protective agent, and the protective agent includes at least one of an alkaloid, an amino acid and a bioalcohol. In some embodiments, the protective agent includes an alkaloid and an amino acid. In some embodiments, the protective agent includes an alkaloid, a bioalcohol, and an amino acid.

In some embodiments, the alkaloid is a betaine. In some embodiments, a molar ratio of the alkaloid to the NaCl is 0.02-0.16:1. Optionally, the molar ratio of the alkaloid to the NaCl is 0.034:1, 0.058:1, 0.077:1, 0.092:1, 0.126:1, or 0.149:1.

In some embodiments, the amino acid is proline. In some embodiments, a molar ratio of the amino acid to the NaCl is 0.005-0.1:1. Optionally, the molar ratio of the amino acid to the NaCl is 0.018:1, 0.035:1, 0.063:1, 0.086:1, or 0.097:1.

In some embodiments, the bioalcohol is mannitol. In some embodiments, a molar ratio of the bioalcohol to the NaCl is 0.005-0.1:1. Optionally, the molar ratio of the alkaloid to the NaCl is 0.018:1, 0.035:1, 0.063:1, 0.086:1, or 0.097:1.

In some embodiments, the centrifugation treatment in step S2 is performed at a speed of 3500-4500 rpm for 30-70 s.

In some embodiments, an isolation and extraction device is used in the extraction method to implement the extraction of an extracellular secretory membrane structure.

In some embodiments, the isolation and extraction device includes a rotation center and at least one extraction mechanism. The extraction mechanism includes a sample injection unit, a capturing and releasing unit, and an enrichment unit. The sample injection unit is configured to input and store a biological sample. The surface-modified membrane is provided in the capturing and releasing unit, and the capturing and releasing unit is connected to the sample injection unit and located at a downstream side of the sample injection unit to capture or release a target extract in the biological sample. The enrichment unit is connected to the capturing and releasing unit and located at a downstream side of the capturing and releasing unit to enrich the target extract released by the capturing and releasing unit.

When the isolation and extraction device is used to isolate and extract an extracellular secretory membrane structure in a biological sample, the biological sample is first added to the sample injection unit of the isolation and extraction device, and then flows into the capturing and releasing unit. The extracellular secretory membrane structure in the biological sample is captured on the surface-modified membrane provided in the capturing and releasing unit. The captured extracellular secretory membrane structure is then released from the surface-modified membrane, and is finally enriched in the enrichment unit, thereby obtaining a collection fluid containing the extracellular secretion membrane structure with high purity and high concentration.

In some embodiments, a central part of the separation device is configured as a rotation mounting part, and the rotation center of the isolation and extraction device serves as a rotation center during a centrifugation operation. In some embodiments, one or more, such as two, three, four, five or six, extraction mechanisms may be provided.

In some embodiments, the sample injection unit includes a sample pool and a sample inlet formed in the sample pool, the sample inlet is connected to an internal cavity of the sample pool, and the sample pool is configured to store the biological sample.

In some embodiments, the sample injection unit further includes a sample quantification pool and a redundant sample pool, a first channel is provided between the sample pool and the sample quantification pool to connect the sample pool and the sample quantification pool, and a second channel is provided between the sample quantification pool and the redundant sample pool to connect the sample quantification pool and the redundant sample pool. When the isolation and extraction device is used to isolate and extract an extracellular secretory membrane structure from a biological sample, the biological sample is first added to the sample pool, and then flows into the sample quantification pool. An excess amount of biological sample is generally added and redundant part of the biological sample flows into the redundant sample pool. The amount of the biological sample treated each time is the maximum volume of the sample quantification pool.

In some embodiments, the first channel is configured as a capillary channel. Since the first channel is configured as a capillary channel, the biological sample in the sample pool can enter the sample quantification pool more smoothly and quickly under the siphon action.

In some embodiments, the sample injection unit further includes a first impurity collection pool, and a third channel is provided between the first impurity collection pool and the sample quantification pool to connect the first impurity collection pool and the sample quantification pool. In some embodiments, the first impurity collection pool is provided at a side of the sample quantification pool away from the rotation center. In this embodiment, large molecular weight impurities in the sample can be easily removed under the action of centrifugal force during centrifugation, so that the impurities are collected in the first impurity collection pool.

In some embodiments, the third channel is configured as a necked channel; a side of the sample quantification pool connected to the first impurity collection pool has a gradually rising structure, which may gradually rise in a step-like manner, or may gradually rise in a slope-like manner. By means of the above structural arrangement, the impurities in the sample quantification pool can enter the first impurity collection pool more smoothly under the action of centrifugal force, and the purified biological sample can enter the capturing and releasing unit.

In some embodiments, a liquid outlet end of the sample quantification pool is connected to a liquid inlet end of the capturing and releasing unit.

In some embodiments, the isolation and extraction device further includes a plurality of weight-reduction balancing pools, and an air channel is provided between the redundant sample pool and one of the weight-reduction balancing pools to connect the redundant sample pool and the weight-reduction balancing pool. Air holes are provided on the bottom plate at positions corresponding to the weight-reduction balancing pools so that gas generated during centrifugation can be discharged at any time. In this way, the isolation and extraction device is connected to the outside atmosphere.

In some embodiments, at least one of the weight-reduction balancing pools connected to the redundant sample pool is provided with an air hole.

In some embodiments, the capturing and releasing unit includes a loading solution pool, an eluent pool, an incubation pool, and a carrier filter pool; the incubation pool is connected to liquid outlet ends of the sample injection unit and the loading solution pool respectively, the carrier filter pool is connected to liquid outlet ends of the incubation pool and the eluent pool respectively, and the surface-modified membrane is provided in the carrier filter pool; the loading solution pool, the incubation pool, and the carrier filter pool are distributed in sequence from a proximal end of the rotation center to its distal end.

In some embodiments, the incubation pool is connected to the liquid outlet end of the sample quantification pool.

The loading solution pool may be configured to add the loading solution, and the eluent pool may be configured to add the eluent. In some embodiments, one or more eluent pools are provided, and the types of eluents added into the multiple eluent pools may be the same or different.

In some embodiments, a fourth channel is provided between the loading solution pool and the incubation pool to connect the loading solution pool and the incubation pool. In some embodiments, the incubation pool is provided at a side of the loading solution pool away from the rotation center; or the incubation pool and the loading solution pool are provided on a same radial line.

In some embodiments, the capturing and releasing unit further includes a mixed solution transfer channel connecting the incubation pool and the carrier filter pool. In some embodiments, the mixed solution transfer channel is configured as an arc-shaped channel to facilitate the transfer of the liquid contained therein under the action of centrifugal force.

In some embodiments, a fifth channel is also provided between the mixed solution transfer channel and the incubation pool. In some embodiments, the fifth channel has a smaller inner diameter than the mixed solution transfer channel. In some embodiments, the fifth channel is configured as a capillary channel, so that the liquid in the incubation pool can smoothly flow to the mixed solution transfer channel under the siphon action of the capillary channel. In one example, the mixed solution transfer channel is configured as an arc-shaped channel. This structural arrangement allows the liquid to enter next treatment unit under the action of centrifugal force.

In some embodiments, the carrier filter pool includes a first membrane placement chamber and a mixed solution chamber, the mixed solution chamber is provided at a side close to the rotation center relative to the first membrane placement chamber, a first filter assembly is provided in the first membrane placement chamber, and the first filter assembly includes the surface-modified membrane.

In some embodiments, the first membrane placement chamber has a greater depth than the mixed solution chamber, so that the liquid in the mixed solution chamber can enter the first membrane placement chamber under the action of gravity.

In some embodiments, the first filter assembly is inserted into the first membrane placement chamber.

In some embodiments, the first filter assembly includes a first membrane base plate and the surface-modified membrane, the first membrane base plate has a window in the middle for liquid to pass through, and the surface-modified membrane is provided at a side of the first membrane base plate close to the rotation center. The first membrane base plate is configured to support the surface-modified membrane, thereby allowing the surface-modified membrane to be inserted into the first membrane placement chamber.

After both the loading solution and the sample from the sample quantification pool enter the incubation pool, the loading solution and the sample are mixed in the incubation pool, and the resulting mixed solution then enters the mixed solution chamber of the carrier filter pool, and then passes through the surface-modified membrane provided in the first membrane placement chamber. In this way, the substances in the biological sample, including the extracellular secretion membrane structure, are trapped on the surface-modified membrane, and others pass through the surface-modified membrane and then flow out from a hollow portion of the membrane base plate.

In some embodiments, a side of the carrier filter pool away from the rotation center is a plane perpendicular to the bottom of the pool. In some embodiments, the side of the carrier filter pool away from the rotation center gradually rises from the bottom of the pool to the surface of the pool. It may gradually rise from the bottom of the pool to the surface of the pool in a step-like manner or may gradually rise from the bottom of the pool to the surface of the pool in a slope-like manner. The gradually rising arrangement facilitates the transfer of liquid from a previous treatment pool to next treatment pool.

In some embodiments, the enrichment unit includes a target extract pool and a waste liquid pool that are respectively connected to the carrier filter pool. The target extract pool is configured to collect the target extract from the carrier filter pool, and the waste liquid pool is configured to collect waste liquid from the carrier filter pool.

In some embodiments, a sixth channel is provided between the carrier filter pool and the waste liquid pool, so that the waste liquid flowing out of the carrier filter pool enters the waste liquid pool via the sixth channel. In some embodiments, the sixth channel is configured as a linear channel. In some embodiments, the sixth channel is configured as a curved channel, and the arrangement of the curved channel is conducive to increasing the path of the channel.

In some embodiments, a seventh channel is provided between the carrier filter pool and the target extract pool, so that the target extract flowing out of the carrier filter pool enters the target extract pool via the seventh channel. In some embodiments, a liquid inlet end of the seventh channel is connected to the sixth channel. In some embodiments, connected ends of the seventh channel and the sixth channel are arranged at an acute angle or a right angle. In some embodiments, the connected ends of the seventh channel and the sixth channel form an included angle of 30-90°, for example, 37°, 42°, 48°, 56°, 64°, 72°, 79°, or 84°. The seventh channel and the sixth channel are arranged at an acute angle or a right angle, which facilitates the transfer of the target extract to the target extract pool. In addition, when the seventh channel and the sixth channel are arranged at an acute angle or a right angle and the sixth channel is configured as a curved channel, less eluent containing the target extract enters the waste liquid pool, and the loss of the target extract can be reduced.

In some embodiments, the eluent pool is provided at a side of the carrier filter pool close to the rotation center. In some embodiments, an eighth channel is provided between the eluent pool and the carrier filter pool to connect the eluent pool and the carrier filter pool.

In some embodiments, the target extract pool and the waste liquid pool are provided on a same side or on opposite sides with respect to the sixth channel.

In some embodiments, the target extract pool and at least one weight-reduction balancing pool are connected by an air channel; an air hole is provided on the bottom plate at a position corresponding to the at least one weight-reduction balancing pool that is connected to the target extract pool, so that gas generated during centrifugation can be discharged at any time. In this way, the isolation and extraction device is connected to the outside atmosphere.

In some embodiments, the eluent pool is configured as a circular pool or a polygonal pool, such as a square pool, a pentagonal pool, or a hexagonal pool.

In some embodiments, the eluent pool further includes an eluent container assembly provided in the eluent pool, the eluent container assembly includes an eluent container and a first container lid, and the first container lid matches the eluent container in shape; when the eluent container and the first container lid are placed in the eluent pool, an upper surface of the first container lid and an upper surface of the eluent pool are substantially flush; a first ejector pin is provided at the bottom of the eluent pool close to the eighth channel, a first liquid release hole is provided at the bottom of the eluent container, and a first sealing film is also provided at the bottom of the eluent container to seal the ejector pin hole.

Based on the above arrangement, the eluent may be placed in the eluent container in advance, and during extraction, the first sealing film is broken by the first ejector pin to release the eluent from the eluent container, and the eluent then flows out from the eighth channel. This embodiment avoids an additional step of adding the eluent and can realize the isolation and extraction of the target extract in one go just by adding a biological sample.

In some embodiments, a first support platform is provided on the bottom of the eluent pool and configured to support the eluent container. When the first ejector pin pushes the eluent container by means of the first liquid release hole, the first support platform serves as the fulcrum of the eluent container to take a leverage effect so that the first ejector pin breaks the first sealing film on the eluent container to release the eluent.

In some embodiments, the first sealing film is provided on an inner side or outer side of the bottom of the eluent container.

In some embodiments, a ninth channel is provided on an upper surface of the first ejector pin and points to the eighth channel. This arrangement helps the eluent from the eluent container flow to the eighth channel.

In some embodiments, a first bump is provided on a side wall of the eluent pool, and a first recessed wall is provided on an outer side of the eluent container, and the first bump matches the first recessed wall. Based on this arrangement, the first ejector pin is located above the first liquid release hole, and it can be ensured that the first ejector pin breaks the first sealing film timely during centrifugation, allowing the eluent to flow out from the first ejector pin hole. Further, a first notch that matches the first bump is formed at a corresponding position on the first container lid. In some embodiments, the first bump may be shaped as a curved bump or a polygonal bump, or the first bump may be in any shape that can make the position of the eluent container relatively fixed.

In some embodiments, the structural arrangement of the eluent pool is similar to that of the loading solution pool.

In some embodiments, the loading solution pool is configured as a circular pool or a polygonal pool, such as a square pool, a pentagonal pool, or a hexagonal pool.

In some embodiments, the loading solution pool further includes a loading solution container assembly provided in the loading solution pool, the loading solution container assembly includes a loading solution container and a second container lid, and the second container lid matches the loading solution container in shape; when the loading solution container and the second container lid are placed in the loading solution pool, an upper surface of the second container lid and an upper surface of the loading solution pool are substantially flush; a second ejector pin is provided at the bottom of the loading solution pool close to the fourth channel, a second liquid release hole is provided at the bottom of the loading solution container, and a second sealing film is also provided at the bottom of the loading solution container to seal the second liquid release hole.

Based on the above arrangement, the loading solution may be pre-embedded in the loading solution container, and during extraction, the second sealing film is broken by the second ejector pin to release the loading solution from the loading solution container, and the loading solution then flows out from the fourth channel. This embodiment avoids an additional step of adding the loading solution and can realize the isolation and extraction of the target extract in one go just by adding a biological sample.

In some embodiments, a second support platform is provided on the bottom of the loading solution pool and configured to support the loading solution container. When the second ejector pin pushes the loading solution container by means of the second liquid release hole, the second support platform serves as the fulcrum of the loading solution container to take a leverage effect so that the second ejector pin breaks the second sealing film on the loading solution container to release the loading solution.

In some embodiments, the second sealing film is provided on an inner side or outer side of the bottom of the loading solution container.

In some embodiments, a tenth channel is provided on an upper surface of the second ejector pin and points to the fourth channel. This arrangement helps the loading solution from the loading solution container flow to the fourth channel.

In some embodiments, a second bump is provided on a side wall of the loading solution pool, and a second recessed wall is provided on an outer side of the loading solution container, and the second bump matches the second recessed wall. Based on this arrangement, the second ejector pin is located above the second liquid release hole and it can be ensured that the second ejector pin breaks the second sealing film timely during centrifugation, allowing the loading solution to flow out from the second liquid release hole. Further, a second notch that matches the second bump is formed at a corresponding position on the second container lid.

In some embodiments, the first sealing film is an aluminum plastic film; the second sealing film is an aluminum plastic film.

In some embodiments, the isolation and extraction device further includes a sample pretreatment unit, the sample pretreatment unit includes an impurity filter pool, the impurity filter pool is connected to the incubation pool, and the microfiltration membrane for trapping impurities is provided in the impurity filter pool.

In some embodiments, the structural arrangement of the impurity filter pool is similar to that of the carrier filter pool.

In some embodiments, the impurity filter pool includes a second membrane placement chamber and a quantification liquid chamber, the quantification liquid chamber is provided at a side close to the rotation center relative to the second membrane placement chamber, a second filter assembly is provided in the second membrane placement chamber, and the second filter assembly includes the microfiltration membrane.

In some embodiments, the second membrane placement chamber has a greater depth than the quantification liquid chamber, so that the liquid in the quantification liquid chamber can enter the second membrane placement chamber under the action of gravity.

In some embodiments, the second filter assembly is inserted into the second membrane placement chamber.

In some embodiments, the second filter assembly includes a second membrane base plate and the microfiltration membrane, the second membrane base plate is hollow in the middle, and the microfiltration membrane is provided at a side of the second membrane base plate close to the rotation center. The second membrane base plate is configured to support the microfiltration membrane, thereby allowing the microfiltration membrane to be inserted into the second membrane placement chamber.

In some embodiments, an eleventh channel is provided between the impurity filter pool and the sample quantification pool to connect the impurity filter pool and the sample quantification pool. In some embodiments, a liquid inlet end of the eleventh channel is connected to the third channel. In some embodiments, the eleventh channel is configured as a capillary channel, so that the solution in the sample quantification pool is siphoned into the impurity filter pool under the siphon action of the eleventh channel.

In some embodiments, a twelfth channel is provided between the impurity filter pool and the incubation pool to connect the impurity filter pool and the incubation pool.

After completely entering the impurity filter pool, the sample from the sample quantification pool first passes through the quantification liquid chamber, and then passes through the microfiltration membrane provided in the second membrane placement chamber, so that the large molecular weight and/or large-sized substances in the biological sample are trapped on the microfiltration membrane and others pass through the microfiltration membrane and then flow out from the hollow portion of the second membrane base plate.

In some embodiments, a side of the impurity filter pool away from the rotation center is a plane perpendicular to the bottom of the pool. In some embodiments, the side of the impurity filter pool away from the rotation center gradually rises from the bottom of the pool to the surface of the pool. It may gradually rise from the bottom of the pool to the surface of the pool in a step-like manner or may gradually rise from the bottom of the pool to the surface of the pool in a slope-like manner. The gradually rising arrangement facilitates the transfer of liquid from a previous treatment pool to next treatment pool.

In some embodiments, the sample pretreatment unit further includes a second impurity collection pool, and a thirteenth channel is provided between the impurity filter pool and the second impurity collection pool to connect the impurity filter pool and the second impurity collection pool.

In some embodiments, the isolation and extraction device includes a bottom plate and a cover plate that are of matching arrangement, and the treatment pools, the channels, the air channels, the chambers and the air holes are formed on the bottom plate.

The “matching arrangement” refers to matching in appearance and matching of the mounting holes and air holes in position, shape and size.

In some embodiments, the rotation center, ejector pin holes and air holes are provided at corresponding positions on the cover plate. For example, a first ejector pin hole is provided on the cover plate at a position corresponding to the first ejector pin; a second ejector pin hole is provided on the cover plate at a position corresponding to the second ejector pin.

In some embodiments, a main body of the isolation device may be made from a polymer material such as polymethyl methacrylate (PMMA), polycarbonate (PC), and polyvinyl chloride (PVC). Optionally, the main body of the isolation device may be made from polycarbonate (PC). In some embodiments, the bottom plate and the cover plate may be made from a polymer material such as PMMA, PC, and PVC. Optionally, the bottom plate and the cover plate may be made from PC.

In some embodiments, the bottom plate and the cover plate may be bonded by a double-sided adhesive or by heat pressure. In some embodiments, the eluent container and the first container lid may be bonded by a double-sided adhesive or by heat pressure. In some embodiments, the loading solution container and the second container lid may be bonded by a double-sided adhesive or by heat pressure. In addition to using the same polymer material as the bottom plate, the eluent container and the loading solution container may also be coated with an adhesive film on one side, and adhesive layers are hydrophobic. Each of the channels may be hydrophilically modified after being treated with a modifier, so that the liquid can flow out smoothly. In some embodiments, the base plate has a thickness of 3-8 mm, for example, the thickness may be 4 mm, 4.5 mm, 5 mm, and 5.5 mm.

In some embodiments, when the isolation and extraction device described above is used for extracting a target extract from a living organism, the loading solution container assembly is added to the interior of the loading solution pool of the isolation and extraction device, and a loading solution is added into the loading solution container assembly. The surface-modified member is provided in the first membrane placement chamber of the carrier filter pool; the microfiltration membrane is provided in the second membrane placement chamber of the impurity filter pool. The eluent container assembly is provided in the eluent pool, and the eluent pool is added to the interior of the eluent container assembly. A method for extracting a target extract from a living organism using the isolation and extraction device described above includes the following steps:
I. adding the biological sample into the sample pool and controlling the isolation and extraction device to rotate rapidly at a first rotation speed, so that the sample quantification pool is full filled with the biological sample and at the same time, some impurities enter the first impurity collection pool;
II. allowing the biological sample to stand still for a period of time, during which the biological sample gradually enters the impurity filter pool under the siphon action of the eleventh channel and at the same time, the second ejector pin breaks the second sealing film, causing the loading solution to flow out of the loading solution pool;
III. controlling the isolation and extraction device to rotate rapidly at a second rotation speed, so that the biological sample entering the impurity filter pool passes through the microfiltration membrane and then enters the incubation pool and at the same time, the loading solution flowing out of the loading solution pool also enters the incubation pool, and then the biological sample and the loading solution are mixed in the incubation pool;
IV. controlling the isolation and extraction device to rotate in a high-low-high speed cycling pattern to promote the accelerated mixing of the two solutions in the incubation pool;
V. stopping the isolation and extraction device to allow the mixed solution to stand still for a period of time so that the fifth channel is full filled with the mixed solution;
VI. controlling the isolation and extraction device to rotate at a high speed, so that the mixed solution enters the carrier filter pool and passes through the surface-modified membrane and consequently the extracellular secretion membrane structure in the mixed solution is trapped on the surface-modified membrane and the waste liquid enters the waste liquid pool;
VII. stopping the isolation and extraction device to allow the solution stand still for a period of time, during which the first ejector pin breaks the first sealing film, causing the eluent to flow out of the eluent pool; and
VIII. controlling the isolation and extraction device to rotate at a high speed, so that the eluent, after passing through the surface-modified membrane, elutes the extracellular secretion membrane structure from the surface-modified membrane, and then enters the target extract pool.

In some embodiments, the first rotation speed is within a range of 2500-5500 rpm. For example, the first rotation speed may be 2750 rpm, 3000 rpm, 3250 rpm, 3500 rpm, 3750 rpm, 4000 rpm, 4250 rpm, 4500 rpm, 4750 rpm, 5000 rpm, or 5250 rpm. A duration of the first rotation speed is within a range of 120-480 s. For example, the duration of the first rotation speed may be 155 s, 185 s, 235 s, 275 s, 335 s, 395 s, 415 s, or 475 s.

In some embodiments, in step II, a time of standing still is within a range of 5-50 s. For example, the time of standing still is 9 s, 17 s, 23 s, 29 s, 32 s, 38 s, 43 s, or 47 s.

In some embodiments, the second rotation speed is within a range of 2000-4500 rpm. For example, the second rotation speed may be 2250 rpm, 2500 rpm, 2750 rpm, 3000 rpm, 3250 rpm, 3500 rpm, 3750 rpm, 4000 rpm, or 4250 rpm. A duration of the second rotation speed is within a range of 15-120 s. For example, the duration of the second rotation speed may be 25 s, 45 s, 75 s, 95 s, or 115 s.

In some embodiments, in step IV, the high speed is within a range of 2500-4000 rpm; for example, the high speed may be 2750 rpm, 3000 rpm, 3250 rpm, 3500 rpm, or 3750 rpm; a duration of the high speed is within a range of 3-20 s; for example, the duration of the high speed may be 5 s, 8 s, 12 s, 16 s, or 19 s. In some embodiments, in step IV, the low speed is within a range of 300-2000 rpm; for example, the low speed may be 450 rpm, 800 rpm, 1250 rpm, 1500 rpm, 1750, or 1900 rpm; a duration of the low speed is within a range of 3-20 s; for example, the duration of the low speed may be 5 s, 8 s, 12 s, 16 s, or 19 s. In some embodiments, the rotation at the high speed and the rotation at the low speed may be in the same direction or in opposite directions. In some embodiments, the number of high-low-high speed alternating operations may be between 3 and 15, for example, 5, 8, 11, or 13.

In some embodiments, a time of standing still in step V may be within a range of 5-50 s, for example, 15 s, 22 s, 28 s, 34 s, 41 s or 47 s.

In some embodiments, in step VI, the high speed is within a range of 2000-5000 rpm; for example, the high speed may be 2250 rpm, 2500 rpm, 2750 rpm, 3000 rpm, 3250 rpm, 3500 rpm, 3750 rpm, 4000 rpm, 4250 rpm, 4500 rpm, or 4750 rpm; a duration of the high speed is within a range of 20-120 s; for example, the duration of the high speed may be 35 s, 65 s, 85 s, 105 s, or 115 s.

In some embodiments, in step VII, a time of standing still is within a range of 5-40 s. For example, the time of standing still is 10 s, 17 s, 22 s, 29 s, or 37 s.

In some embodiments, in step VIII, the high speed is within a range of 2500-6500 rpm; for example, the high speed may be 2750 rpm, 3350 rpm, 3950 rpm, 4550 rpm, 4900 rpm, 5450 rpm, or 5900 rpm; a duration of the high speed is within a range of 30-120 s; for example, the duration of the high speed may be 35 s, 65 s, 85 s, 105 s, or 115 s.

In some embodiments, the present application also provides an isolation and extraction kit for an extracellular secretory membrane structure. The kit includes the isolation and extraction device which contains the surface-modified membrane and is pre-embedded with the loading solution and the eluent. In some embodiments, the isolation and extraction device further contains the microfiltration membrane.

In some embodiments, the isolation and extraction device is configured as a centrifugal adsorption column. The isolation and extraction device includes a first outer tube having one end open and a first inner tube having two ends open and fitted in the first outer tube. The other end of the first outer tube is closed. A certain receiving space is defined between an end of the first inner tube close to the closed end of the first outer tube and the closed end of the first outer tube. The surface-modified membrane is provided at the end of the first inner tube close to the closed end of the first outer tube.

In some embodiments, the open end of the first outer tube is provided with a first closing cap.

In some embodiments, the end of the first inner tube close to the closed end of the first outer tube is provided with a liquid outlet tube, and the liquid outlet tube has a smaller inner diameter than the first inner tube.

In some embodiments, at least two centrifugal adsorption columns are provided. A surface-modified membrane is provided in the first inner tube of at least one of the centrifugal adsorption columns, and a microfiltration membrane is provided in the first inner tube of at least one of the centrifugal adsorption columns.

When the isolation and extraction device described above is used to isolate and extract an extracellular secretory membrane structure, the following steps are included:
S1. adding a biological sample into the first inner tube of the centrifugal adsorption column provided with the microfiltration membrane, then performing centrifugation, and taking a filtrate for later use;
S2. mixing the filtrate and the loading solution, placing the resulting mixed solution in the first inner tube of the centrifugal adsorption column provided with the surface-modified membrane, then allowing the mixed solution to stand still until all the filtrate passes through the surface-modified membrane, and then discarding the filtrate; and
S3. adding the eluent into the first inner tube provided with the surface-modified membrane; after all the eluent passes through the surface-modified membrane, performing centrifugation and then discarding supernatant to obtain a target sample.

In some embodiments, in step S1, the centrifugation is performed at a speed of 2000-5000 rpm, for example, at 2250 rpm, 2500 rpm, 2750 rpm, 3000 rpm, 3250 rpm, 3500 rpm, 3750 rpm, 4000 rpm, 4250 rpm, 4500 rpm, or 4750 rpm; a time of the centrifugation is within a range of 20-120 s; for example, the time of the centrifugation may be 35 s, 65 s, 85 s, 105 s, or 115 s.

In some embodiments, in step S3, the centrifugation is performed at a speed of 2500-6500 rpm, for example, at 2750 rpm, 3350 rpm, 3950 rpm, 4550 rpm, 4900 rpm, 5450 rpm, or 5900 rpm; a duration of the high speed is within a range of 30-120 s; for example, the duration of the high speed may be 35 s, 65 s, 85 s, 105 s, or 115 s.

The isolation and extraction kit includes at least one centrifugal adsorption column described above, and a surface-modified membrane is provided in the at least one centrifugal adsorption column. The kit further includes a loading solution and an eluent. Further, the isolation and extraction kit includes at least two centrifugal adsorption columns described above, a surface-modified membrane is provided in at least one of the centrifugal adsorption columns, and a microfiltration membrane is provided in at least one of the centrifugal adsorption columns.

In some embodiments, the isolation and extraction device includes at least one syringe, and a needle-holding end of the at least one syringe is provided with a surface-modified membrane.

In some embodiments, the isolation and extraction device includes at least two syringes, a needle-holding end of at least one of the syringes is provided with a surface-modified membrane, and a needle-holding end of at least one of the syringes is provided with a microfiltration membrane.

When the isolation and extraction device described above is used to isolate and extract an extracellular secretory membrane structure, the following steps are included:
S1. adding a biological sample into the syringe provided with the microfiltration membrane and taking a filtrate for later use;
S2. mixing the filtrate and the loading solution, placing the resulting mixed solution in the syringe provided with the surface-modified membrane, then allowing the mixed solution to stand still until all the mixed solution passes through the surface-modified membrane, and then discarding the filtrate; and
S3. adding the eluent into the syringe provided with the surface-modified membrane; after all the eluent passes through the surface-modified membrane, performing centrifugation on the eluent and then discarding supernatant to obtain a target sample.

In some embodiments, in step S1, let the biological sample stand still so that the biological sample passes through the microfiltration membrane; alternatively, a piston rod is pressed down so that the biological sample passes through the microfiltration membrane.

In some embodiments, in step S3, the centrifugation is performed at a speed of 2500-6500 rpm, for example, at 2750 rpm, 3350 rpm, 3950 rpm, 4550 rpm, 4900 rpm, 5450 rpm, or 5900 rpm; a duration of the high speed is within a range of 30-120 s; for example, the duration of the high speed may be 35 s, 65 s, 85 s, 105 s, or 115 s.

The isolation and extraction kit includes at least one syringe described above, and a surface-modified membrane is provided in the at least one syringe. The kit further includes a loading solution and an eluent. Further, the isolation and extraction kit includes at least two syringes described above, a surface-modified membrane is provided in at least one of the syringes, and a microfiltration membrane is provided in at least one of the syringes.

In some embodiments, the syringe may also be replaced with a solid phase extraction column.

The present application will be further described in detail below with reference to the accompanying drawings and embodiments.

### Preparation of the Surface-Modified Membrane

### Preparation Example 1 of the Surface-Modified Membrane

8 mM dodecyl trimethyl ammonium bromide (DTAB) was dissolved in 10 mL of methanol, and the resulting solution was well mixed at room temperature to fully dissolve DTAB; a regenerated cellulose membrane (RC membrane, purchased from sartorius/Whatman) was placed in the solution and reaction was allowed with shaking at room temperature for 40 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Preparation Example 2 of the Surface-Modified Membrane

8.6 mM cetyl trimethyl ammonium bromide (CTAB) was dissolved in 10 mL of methanol, and the resulting solution was well mixed at room temperature to fully dissolve CTAB; an RC membrane was placed in the solution and reaction was allowed with shaking at room temperature for 48 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Preparation Example 3 of the Surface-Modified Membrane

5.6 mM quaternary ammonium salt Gemini surfactant (a Gemini cationic surfactant purchased from Wuhan Huaxiang Kejie Biotechnology Co., Ltd.) was dissolved in 10 mL of 45° C. water, and the resulting solution was well mixed to fully dissolve the quaternary ammonium salt Gemini surfactant; an RC membrane was placed in the solution and reaction was allowed with shaking at room temperature for 54 h; the membrane was then dried in a vacuum drying oven at 60° C. to obtain a quaternary ammonium-modified filter membrane. The quaternary ammonium-modified filter membrane was stored at room temperature for later use.

### Preparation Example 4 of the Surface-Modified Membrane

Preparation of an asymmetric quaternary ammonium salt Gemini surfactant included the following steps.

N,N-dimethyl-n-hexylamine and 1,3-dibromoisopropanol were mixed at a molar ratio of 1:4, absolute ethanol was then added, and the resulting mixed solution was stirred at 70° C. for 12 h and then cooled. After the ethanol was removed by rotary evaporation, diethyl ether was added for extraction, and the extraction solution was then allowed to stand still for 10 min to collect viscous liquid in the lower layer. After filtration, the filtrate was rotary evaporated to remove diethyl ether to obtain an intermediate product 2-hydroxy-3-bromohexane ammonium bromide. 2-hydroxy-3-bromohexyl ammonium bromide and N,N-dimethyloctadecylamine were mixed at a molar ratio of 1:1 to react at 80° C. for 24 h. After the reaction system was cooled, ethanol was removed by rotary evaporation. Then, the filtrate was obtained by recrystallization using a mixed solvent of ethanol/ethyl acetate (v/v: 1:10), and the filtrate was then recrystallized three times using a mixed solvent of ethanol/anhydrous ether (v/v: 1:8) to obtain the final product.

Preparation of the surface-modified membrane included the following steps.

The 3.8 mM asymmetric quaternary ammonium salt Gemini surfactant prepared as above was dissolved in 10 mL of ethanol, and the resulting solution was well mixed to fully dissolve the quaternary ammonium salt Gemini surfactant; an RC membrane was placed in the solution and reaction was allowed with shaking at room temperature for 54 h; the membrane was then dried in a vacuum drying oven at 60° C. to obtain a quaternary ammonium-modified filter membrane. The quaternary ammonium-modified filter membrane was stored at room temperature for later use.

### Preparation Example 5 of the Surface-Modified Membrane

Preparation of a symmetric quaternary ammonium salt Gemini surfactant included the following steps. To a three-necked flask, 0.1 mol of alkyl dimethyl tertiary amine, 0.05 mol of dichloropropane, and 50 mL of ethanol were added and then heated with stirring for refluxing for 48 h. When the reaction solution was cooled for a while and then rotary evaporated to obtain a viscous product. The product was recrystallized using a mixed solution of ethyl acetate. In order to obtain a sufficiently pure product, the product was recrystallized three times using a mixed solution of ethanol/ethyl acetate (v/v: 1:10). The obtained solid product was then dried in a vacuum drying box at 50° C. for 12 h to obtain a symmetrical quaternary ammonium salt Gemini surfactant.

Preparation of the surface-modified membrane included the following steps.

The 4.7 mM symmetric quaternary ammonium salt Gemini surfactant prepared as above was dissolved in 10 mL of ethanol, and the resulting solution was well mixed to fully dissolve the quaternary ammonium salt Gemini surfactant; an RC membrane was placed in the solution and reaction was allowed with shaking at room temperature for 50 h; the membrane was then dried in a vacuum drying oven at 60° C. to obtain a quaternary ammonium-modified filter membrane. The quaternary ammonium-modified filter membrane was stored at room temperature for later use.

### Preparation Example 6 of the Surface-Modified Membrane

5.3 mM trimethoxysilylpropyl trimethyl ammonium chloride (QAS_{CO}, purchased from Macklin) was dissolved in 10 mL of water, and the resulting solution was well mixed and stirred for 2 h to fully dissolve QAS_{C0}; an RC membrane was placed in the solution and reaction was allowed with shaking at room temperature for 24 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Preparation Example 7 of the Surface-Modified Membrane

5.8 mM trimethoxysilylpropyl octadecyldimethyl ammonium chloride (QAS_{C18}, purchased from Macklin) was dissolved in 10 mL of water, and the resulting solution was well mixed and stirred for 2 h to fully dissolve QAS_{C18}; an RC membrane was placed in the solution and reaction was allowed with shaking at room temperature for 24 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Preparation Example 8 of the Surface-Modified Membrane

6.4 mM cocamidopropyl betaine (product model CAB-35, C₁₉H₃₈N₂O₃) was dissolved in 10 mL of water, and the resulting solution was well mixed and stirred to fully dissolve the cocamidopropyl betaine; an RC membrane was placed in the solution and reaction was allowed with shaking at room temperature for 42 h; the membrane was then dried in a vacuum drying oven at 60° C. to obtain a quaternary ammonium-modified filter membrane. The quaternary ammonium-modified filter membrane was stored at room temperature for later use.

### Preparation Example 9 of the Surface-Modified Membrane

3.7 mM octyldecylamidopropyl betaine (product model ODAB-35, available from Wuhan Kangqiong Biomedical Technology Co., Ltd.) was dissolved in 10 mL of ethanol, and the resulting solution was well mixed and stirred to fully dissolve the octyldecylamidopropyl betaine; an RC membrane was placed in the solution and reaction was allowed with shaking at room temperature for 52 h; the membrane was then dried in a vacuum drying oven at 60° C. to obtain a quaternary ammonium-modified filter membrane. The quaternary ammonium-modified filter membrane was stored at room temperature for later use.

### Preparation Example 10 of the Surface-Modified Membrane

4.6 mM 3-(N,N-dimethyldodecyl ammonium) propane sulfonate (SPPT, CAS14933-08-5) was dissolved in 10 mL of methanol, and the resulting solution was well mixed at room temperature for 2 h to fully dissolve SPPT; an RC membrane was placed in the solution and reaction was allowed with shaking at room temperature for 24 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Preparation Example 11 of the Surface-Modified Membrane

0.24 mM DTAB and 6 mM cocamidopropyl betaine (the molar ratio of DTAB to cocamidopropyl betaine was 1.2:3) were dissolved in 10 mL of methanol, and the resulting solution was well mixed at room temperature for 2 h to fully dissolve DTAB and cocamidopropyl betaine; an RC membrane was placed in the solution and reaction was allowed with shaking at room temperature for 48 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Preparation Example 12 of the Surface-Modified Membrane

1.8 mM QAS_{C0} and 4.5 mM SPPT (the molar ratio of QAS_{C0} to SPPT was 1.2:3) were dissolved in 10 mL of methanol, and the resulting solution was well mixed at room temperature for 2 h to fully dissolve QAS_{C0} and SPPT; an RC membrane was placed in the solution and reaction was allowed with shaking at room temperature for 48 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Preparation Example 13 of the Surface-Modified Membrane

1.8 mM QAS_{C18} and 4.5 mM SPPT (the molar ratio of QAS_{C18} to SPPT was 1.2:3) were dissolved in 10 mL of methanol, and the resulting solution was well mixed at room temperature for 2 h to fully dissolve QAS_{C18} and SPPT; an RC membrane was placed in the solution and reaction was allowed with shaking at room temperature for 48 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Preparation Example 14 of the Surface-Modified Membrane

1.8 mM QAS_{C0}, 4.5 mM SPPT (the molar ratio of QAS_{C0} to SPPT was 1.2:3), and 7.6 mM betaine (the molar ratio of the betaine to the surfactant was 1.2:1) were dissolved in 10 mL of methanol, and the resulting solution was well mixed at room temperature for 2 h to fully dissolve the betaine and the surfactant; an RC membrane was placed in the solution and reaction was allowed with shaking at room temperature for 48 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Preparation Example 15 of the Surface-Modified Membrane

1.8 mM QAS_{C0}, 4.5 mM SPPT (the molar ratio of QAS_{C0} to SPPT was 1.2:3), and 2.5 mM proline (the molar ratio of proline to the surfactant was 0.4:1) were dissolved in 10 mL of methanol, and the resulting solution was well mixed at room temperature for 2 h to fully dissolve proline and the surfactant; an RC membrane was placed in the solution and reaction was allowed with shaking at room temperature for 48 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane.

The quaternary ammonium-modified filter membrane was stored at room temperature for later use.

### Preparation Example 16 of the Surface-Modified Membrane

1.8 mM QAS_{C0}, 4.5 mM SPPT (the molar ratio of QAS_{C0} to SPPT was 1.2:3), and 3.8 mM mannitol (the molar ratio of mannitol to the surfactant was 0.6:1) were dissolved in 10 mL of methanol, and the resulting solution was well mixed at room temperature for 2 h to fully dissolve mannitol and the surfactant; an RC membrane was placed in the solution and reaction was allowed with shaking at room temperature for 48 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Preparation Example 17 of the Surface-Modified Membrane

1.8 mM QAS_{C0}, 4.5 mM SPPT (the molar ratio of QAS_{C0} to SPPT was 1.2:3), 7.6 mM betaine and 2.5 mM proline were dissolved in 10 mL of methanol, and the resulting solution was well mixed at room temperature for 2 h to fully dissolve the betaine, the surfactant and proline; an RC membrane was placed in the solution and reaction was allowed with shaking at room temperature for 48 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Preparation Example 18 of the Surface-Modified Membrane

1.8 mM QAS_{C0}, 4.5 mM SPPT (the molar ratio of QAS_{C0} to SPPT was 1.2:3), 7.6 mM betaine, 2.5 mM proline, and 3.8 mM mannitol were dissolved in 10 mL of methanol, and the resulting solution was well mixed at room temperature for 2 h to fully dissolve the betaine, the surfactant, proline and mannitol; an RC membrane was placed in the solution and reaction was allowed with shaking at room temperature for 48 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Preparation Example 19 of the Surface-Modified Membrane

1.8 mM QAS_{C18}, 4.5 mM SPPT (the molar ratio of QAS_{C18} to SPPT was 1.2:3), 7.6 mM betaine, 2.5 mM proline, and 3.8 mM mannitol were dissolved in 10 mL of methanol, and the resulting solution was well mixed at room temperature for 2 h to fully dissolve the betaine, the surfactant, proline and mannitol; an RC membrane was placed in the solution and reaction was allowed with shaking at room temperature for 48 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Preparation Example 20 of the Surface-Modified Membrane

6.4 mM cocamidopropyl betaine (product model CAB-35, C₁₉H₃₈N₂O₃), 7.7 mM betaine, 2.6 mM proline, and 3.8 mM mannitol was added to 10 ml of water, and the resulting solution was well mixed and stirred to fully dissolve the cocamidopropyl betaine; an RC membrane was placed in the solution and reaction was allowed with shaking at room temperature for 42 h; the membrane was then dried in a vacuum drying oven at 60° C. to obtain a quaternary ammonium-modified filter membrane. The quaternary ammonium-modified filter membrane was stored at room temperature for later use.

### Preparation Example 21 of the Surface-Modified Membrane

4.6 mM SPPT, 5.5 mM betaine, 1.8 mM proline, and 2.8 mM mannitol were added to 10 mL of methanol, and the resulting solution was well mixed at room temperature for 2 h to fully dissolve SPPT, the betaine, proline and mannitol; an RC membrane was placed in the solution and reaction was allowed with shaking at room temperature for 48 h; the membrane was then dried in a vacuum drying oven at 60° C.; and the membrane was then rinsed three times with 95% ethanol and then dried in the vacuum drying oven at 60° C. to obtain a quaternary ammonium-modified filter membrane. The quaternary ammonium-modified filter membrane was stored at room temperature for later use.

### Example 1

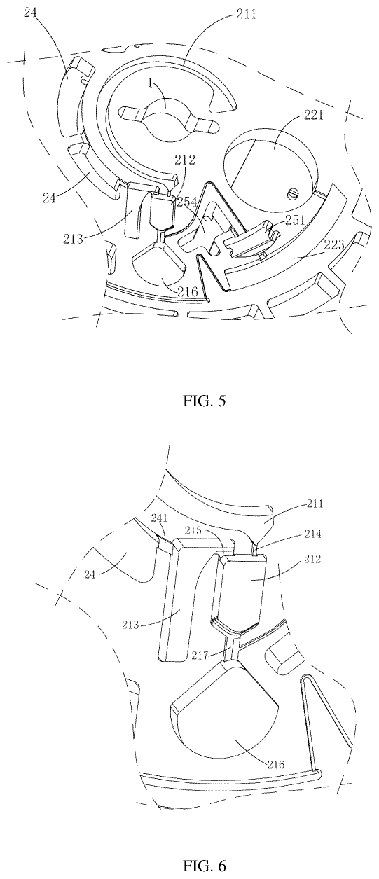

A centrifugal adsorption column 5, as shown in FIG. 5, includes a first outer tube 51

having one end open and a first inner tube 52 having two ends open and fitted in the first outer tube 51. The other end of the first outer tube 51 is closed. A certain receiving space is defined between an end of the first inner tube 52 close to the closed end of the first outer tube 51 and the closed end of the first outer tube 51. A surface-modified membrane 22432 is provided at the end of the first inner tube 52 close to the closed end of the first outer tube 51.

The outer diameter of the first inner tube 52 is the same as the inner diameter of the first outer tube 51 (shown for illustration only), that is, the first inner tube 52 is clamped in the first outer tube 51. The open end of the first outer tube 51 is provided with a first closing cap 511 to prevent liquid from splashing during centrifugation. The structure of the end of the first inner tube 52 close to the closed end of the first outer tube 51 is similar to the design of a needle-holding end of a syringe. The end of the first inner tube 52 close to the closed end of the first outer tube 51 is provided with a liquid outlet tube 521, and the liquid outlet tube 521 has a smaller inner diameter than the first inner tube 52. In this way, it can reduce the probability that liquid coming out of the first inner tube 52 splashes to the side wall of the first outer tube 51, resulting in poor extraction effect. In addition, two centrifugal adsorption columns 5 are provided, i.e., a first centrifugal adsorption column and a second centrifugal adsorption column. The difference between the first centrifugal adsorption column and the second centrifugal adsorption column lies in that: a microfiltration membrane 25132 (not shown) is clamped at one end of the liquid outlet tube 521 of the first inner tube 52 of the first centrifugal adsorption, and a surface-modified membrane 22432 is clamped at one end of the liquid outlet tube 521 of the first inner tube 52 of the second centrifugal adsorption column.

The volumes of the first centrifugal adsorption column and the second centrifugal adsorption column are both 3 ml.

A method for extracting an exosome, implemented using the centrifugal adsorption columns, includes the following steps.

In step S1, 500 μL of whole blood sample was loaded to the first centrifugal adsorption column and then centrifuged at 2500 rpm for 60 s. The filtrate was taken for later use to obtain plasma.

In step S2, the plasma (200 μL) and a loading solution (200 μL for each of them) were mixed at a volume ratio of 1:1, and the resulting mixed solution was then placed in the second centrifugal adsorption column provided therein with the surface-modified membrane 22432. The surface-modified membrane 22432 used in this example was prepared in accordance with the Preparation Example 1 of the surface-modified membrane and was loaded with DTAB. Then, the mixed solution was allowed to stand still until all the mixed solution passed through the surface-modified membrane 22432. The first inner tube 52 was then taken out of the second centrifugal adsorption column 5 with the filtrate being discarded, and then the first inner tube 52 was installed into the first outer tube 51.

In step S3, an eluent was then added to the first inner tube 52 of the second centrifugal adsorption column 5 to elute the surface-modified membrane 22432 loaded with the exosome. The high-concentration salt solution in the eluate caused the exosome to detach from the surface-modified membrane 22432 to obtain an exosome collection fluid.

The main components of the loading solution used were 100 mM tris-propane and 150 mM NaCl, pH 6.5. The main components of the eluent used were 50 mM tris-propane and 1 M NaCl, pH 6.36.

### Example 2

This example is the same as Example 1 except that the surface-modified membrane used in this example was prepared in accordance with the Preparation Example 2 of the surface-modified membrane and was loaded with CTAB; and that
the main components of the loading solution used were 100 mM tris-propane and 150 mM NaCl, pH 6.5; and the main components of the eluent used were 50 mM tris-propane and 2 M NaCl, pH 6.36.

### Example 3

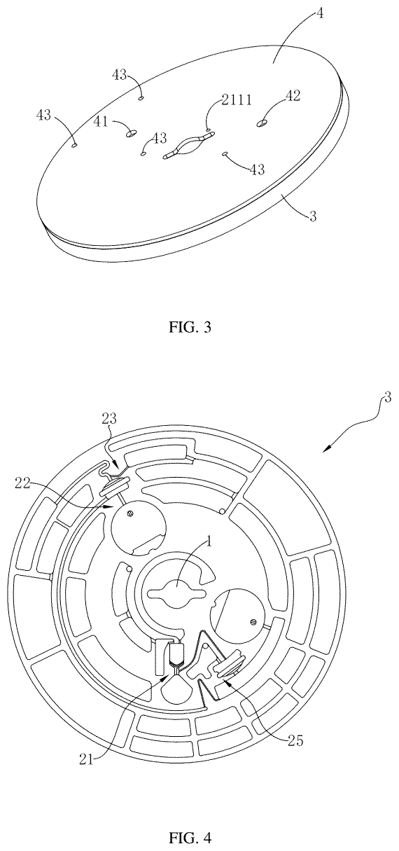

A method for extracting an exosome, implemented using syringes, is provided, where two syringes 6 are used and they are a first syringe and a second syringe, respectively. As shown in FIG. 2, a surface-modified membrane 22432 is fixed in the first syringe at one end close to a needle-holding end; a microfiltration membrane 25132 (not shown) is fixed in the second syringe at one end close to a needle-holding end. The volumes of the first syringe and the second syringe are both 3 mL.

The extraction method includes the following steps.

In step S1, 500 μL of whole blood sample was added to the second syringe. A piston rod of the second syringe was then pressed to allow the whole blood sample to pass through the microfiltration membrane. The filtrate, that is, the plasma, was kept for later use.

In step S2, the plasma (200 μL) and a loading solution (200 μL) were mixed at a volume ratio of 1:1, and the resulting mixed solution was then placed in the first syringe provided therein with the surface-modified membrane. The surface-modified membrane used in this example was prepared in accordance with the Preparation Example 3 of the surface-modified membrane and was loaded with a quaternary ammonium salt Gemini surfactant. Then, the mixed solution was allowed to stand still until all the mixed solution passed through the surface-modified membrane, and the filtrate was discarded.

In step S3, an eluent (300 μL) was then added to the first syringe and a piston rod of the first syringe was pressed to push the eluent to elute the surface-modified membrane loaded with the exosome. The high-concentration salt solution in the eluate caused the exosome to detach from the surface-modified membrane to obtain an exosome collection fluid.

The main components of the loading solution used were 100 mM tris-propane and 168 mM NaCl, pH 6.5. The main components of the eluent used were 50 mM tris-propane and 2.45 M NaCl, pH 6.36.

### Example 4

This example is the same as Example 1 except that the surface-modified membrane used in this example was prepared in accordance with the Preparation Example 4 of the surface-modified membrane and was loaded with an asymmetric quaternary ammonium salt Gemini surfactant; and that the main components of the loading solution used were 100 mM tris-propane and 170 mM NaCl, pH 6.5; and the main components of the eluent used were 50 mM tris-propane and 2.5 M NaCl, pH 6.36.

### Example 5

This example is the same as Example 1 except that the surface-modified membrane used in this example was prepared in accordance with the Preparation Example 5 of the surface-modified membrane and was loaded with a symmetric quaternary ammonium salt Gemini surfactant; and that
the main components of the loading solution used were 100 mM tris-propane and 145 mM NaCl, pH 6.5; and the main components of the eluent used were 50 mM tris-propane and 2.1 M NaCl, pH 6.36.

### Example 6

A method for extracting an exosome, implemented using a separation and extraction device is provided, where a microfluidic centrifuge disk is used as an extraction device. As shown in FIG. 3, the microfluidic centrifuge disk includes a bottom plate 3 and a cover plate 4.

As shown in FIG. 4, a rotation center 1 and an extraction mechanism 2 are provided on the bottom plate 3, and the extraction mechanism 2 includes a sample injection unit 21, a capturing and releasing unit 22 and an enrichment unit 23; a surface-modified membrane 22432 (FIG. 12) is provided in the capturing and releasing unit 22, and the capturing and releasing unit 22 is connected to the sample injection unit 21 and located at the downstream side of the sample injection unit 21 to capture or release a target extract in a biological sample; the enrichment unit 23 is connected to the capturing and releasing unit 22 and located at the downstream side of the capturing and releasing unit 22 to enrich the exosome released from the capturing and releasing unit 22.

A central part of the bottom plate 3 is configured as a rotation mounting part, and the rotation center 1 serves as a rotation center during a centrifugation operation.

As shown in FIG. 3 and FIG. 5, the sample injection unit 21 includes a sample pool 211, a sample inlet 2111 is provided at a corresponding position on the cover plate 4, and a whole blood sample is injected from the sample inlet 2111. As shown in FIG. 5 and FIG. 6, the sample injection unit 21 further includes a sample quantification pool 212 and a redundant sample pool 213; a first channel 214 is provided between the sample pool 211 and the sample quantification pool 212 to connect the sample pool 211 and the sample quantification pool 212, and the first channel 214 is configured as a capillary channel so that the biological sample in the sample pool 211 can enter the sample quantification pool 212 more smoothly and quickly under the siphon action; a second channel 215 is provided between the sample quantification pool 212 and the redundant sample pool 213 to connect the sample quantification pool 212 and the redundant sample pool 213. In addition, the redundant sample pool 213 is connected to one of weight-reduction balancing pools 24 by means of an air channel 241. As shown in FIG. 3, the cover plate 4 is provided with an air hole 43 at a corresponding position so that gas can be discharged timely during the centrifugation process. In sample injection, the biological sample is first added to the sample pool and then flows into the sample quantification pool 212 and redundant part of the sample is allowed to flow into the redundant sample pool 213. The amount of the biological sample treated each time is the maximum volume of the sample quantification pool 212.

The sample injection unit 21 further includes a first impurity collection pool 216, and the first impurity collection pool 216 is provided at a side of the sample quantification pool 212 away from the rotation center 1. A third channel 217 is provided between the first impurity collection pool 216 and the sample quantification pool 212 to connect the first impurity collection pool 216 and the sample quantification pool 212. The third channel 217 is configured as a necked channel and a side of the sample quantification pool 212 connected to the first impurity collection pool 216 is configured in a step-like manner. By this arrangement, impurities in the sample quantification pool 212 can enter the first impurity collection pool 216 more smoothly under the action of centrifugal force.

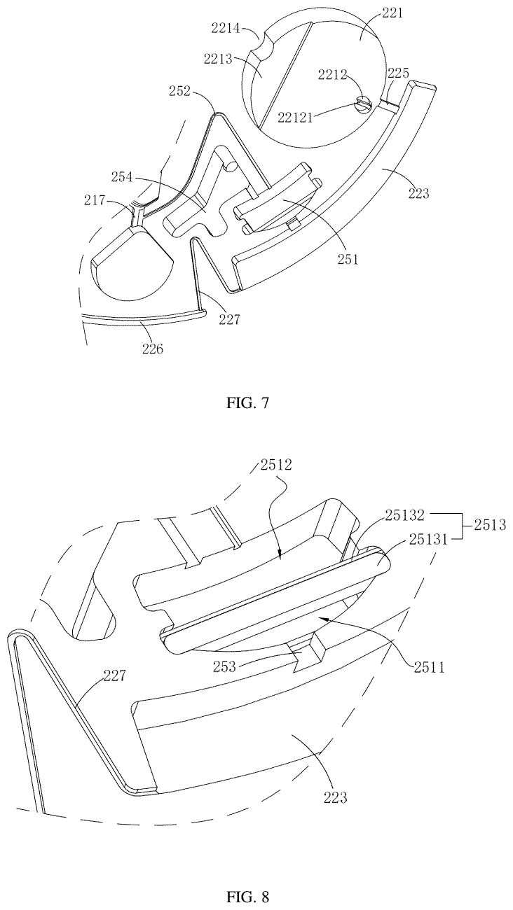

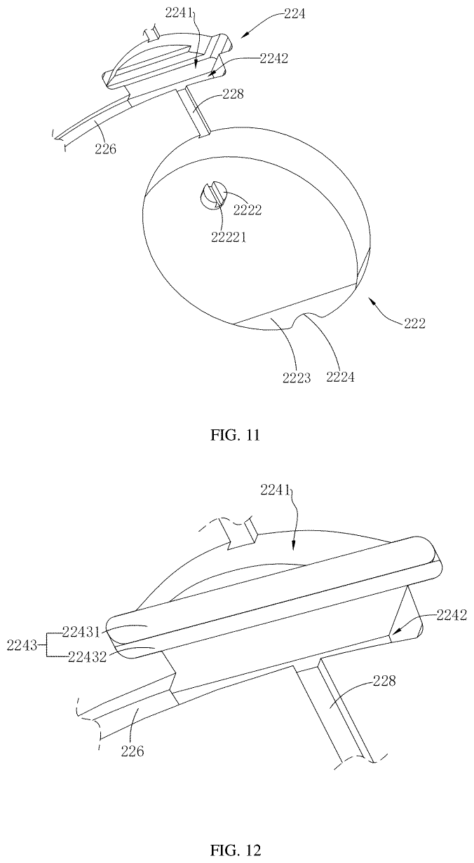

As shown in FIG. 7, the capturing and releasing unit 22 includes a loading solution pool 221, an eluent pool 222 (FIG. 11), an incubation pool 223, and a carrier filter pool 224 (FIG. 11); the incubation pool 223 is connected to liquid outlet ends of the sample injection unit 21 and the loading solution pool 221 respectively, the carrier filter pool 224 is connected to liquid outlet ends of the incubation pool 223 and the eluent pool 222 respectively, and a surface-modified membrane 22432 is provided in the carrier filter pool 224. The surface-modified membrane 22432 used in this example was prepared in accordance with the Preparation Example 6 of the surface-modified membrane and was loaded with QAS_{C0}. In addition, the loading solution pool 221, the incubation pool 223, and the carrier filter pool 224 are distributed in sequence from a proximal end of the rotation center 1 to its distal end.

As shown in FIG. 7, the bottom plate 3 is further provided with a sample pretreatment unit 25 between the sample injection unit 21 and the capturing and releasing unit, the sample pretreatment unit 25 includes an impurity filter pool 251 and a second impurity collection pool 254, the upstream side of the impurity filter pool 251 is connected to the downstream side of the sample quantification pool 212, and the impurity filter pool 251 is also connected to the second impurity collection pool 254. An eleventh channel 252 is provided between the impurity filter pool 251 and the sample quantification pool 212 to connect the impurity filter pool 251 and the sample quantification pool 212, and a liquid inlet end of the eleventh channel 252 is connected to the third channel 217. The eleventh channel 252 is configured as a capillary channel, so that the solution in the sample quantification pool 212 is smoothly siphoned into the impurity filter pool 251.

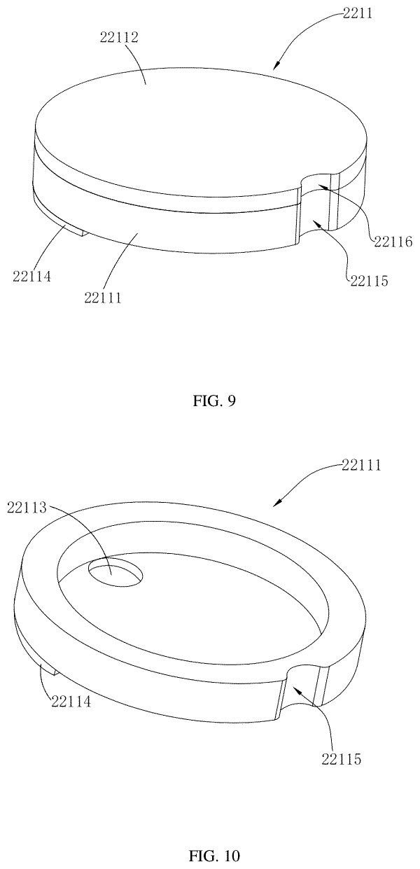

As shown in FIG. 8, the impurity filter pool 251 is provided therein with a second membrane placement chamber 2511 and a quantification liquid chamber 2512, the quantification liquid chamber 2512 is provided at a side close to the rotation center 1 relative to the second membrane placement chamber 2511, and a second filter assembly 2513 is inserted in the second membrane placement chamber 2511. In addition, the second membrane placement chamber 2511 has a greater depth than the quantification liquid chamber 2512, so that the liquid in the quantification liquid chamber 2512 can enter the second membrane placement chamber 2511 under the action of gravity. In addition, the side of the impurity filter pool 251 away from the rotation center 1 is configured in a step-like manner from the bottom of the pool to the surface of the pool. This arrangement facilitates the transfer of liquid from a previous treatment pool to next treatment pool.

The second filter assembly 2513 includes a second membrane base plate 25131 configured to support a microfiltration membrane 25132 and the microfiltration membrane 25132. The second membrane base plate 25131 has a window in the middle for liquid to pass through, and the microfiltration membrane 25132 is adhered to a side of the second membrane base plate 25131 close to the rotation center 1.

A twelfth channel 253 is provided between the impurity filter pool 251 and the incubation pool 223 to connect the impurity filter pool 251 and the incubation pool 223. After completely entering the impurity filter pool 251, the sample from the sample quantification pool 212 first passes through the quantification liquid chamber 2512, and then passes through the microfiltration membrane 25132 provided in the second membrane placement chamber 2511, so that the large molecular weight and/or large-sized substances in the biological sample are trapped on the microfiltration membrane 25132 and others pass through the microfiltration membrane 25132 and then flow out from the hollow portion of the second membrane base plate 25131 to enter the incubation pool 223.

As shown in FIG. 7, the loading solution also enters the incubation pool 223 at the same time, and the loading solution pool 221 is configured to add the loading solution. The loading solution pool 221 is configured as a circular pool. The loading solution pool 221 is located on a side of the incubation pool 223 close to the rotation center 1 and a fourth channel 225 is provided between the loading solution pool 221 and the incubation pool 223 to connect the loading solution pool 221 and the incubation pool 223. The loading solution pool 221 includes a loading solution container assembly 2211 provided in the loading solution pool 221. As shown in FIG. 9, the loading solution container assembly 2211 includes a loading solution container 22111 and a second container lid 22112, and the second container lid 22112 matches the loading solution container in shape; when the loading solution container 22111 and the second container lid 22112 are placed in the loading solution pool 221, an upper surface of the second container lid 22112 and an upper surface of the loading solution pool 221 are substantially flush. Referring to FIG. 7 and FIG. 9, a second ejector pin 2212 is provided at the bottom of the loading solution pool 221 close to the fourth channel; a tenth channel 22121 is provided on an upper surface of the second ejector pin 2212 and points to the fourth channel 225. As shown in FIG. 10, a second liquid release hole 22113 is provided at the bottom of the loading solution container 22111, and a second sealing film 22114 is also provided at the outer side of the bottom of the loading solution container 22111 to seal the second liquid release hole 22113. The second sealing film is an aluminum plastic film and is easy to break. In addition, a second support platform 2213 is provided on the bottom of the loading solution pool 221 and configured to support the loading solution container 22111.

During isolation and extraction, the second ejector pin 2212 pushes the loading solution container 22111 by means of the second liquid release hole 22113, the second support platform 2213 serves as the fulcrum of the loading solution container 22111 to take a leverage effect so that the second ejector pin 2212 breaks the second sealing film 22114 on the loading solution container 22111 to release the loading solution. This solution avoids an additional step of adding the loading solution and can realize the isolation and extraction of the target extract in one go just by adding a biological sample.

In order to prevent the loading solution container assembly 2211 from changing positions randomly in the loading solution pool 221 during centrifugation, a second bump 2214 is provided on a side wall of the loading solution pool 221 and accordingly, a second notch 22116 that matches the second bump 2214 is formed at a corresponding position on the second container lid 22112. A second recessed wall 22115 is provided on an outer side of the loading solution container 22111. After the loading solution container assembly 2211 is placed in the loading solution pool 221, the second bump 2214 is snap connected to the second recess 22116 and the second recessed wall 22115. Based on this arrangement, it can be ensured that the second ejector pin 2212 is located above the second liquid release hole 22113 and that the second ejector pin 2212 breaks the second sealing film 22114 timely during centrifugation, allowing the loading solution to flow out from the second liquid release hole 22114 and enter the carrier filter pool 224.

As shown in FIG. 7, a fifth channel 227 and a mixed solution transfer channel 226 are connected in sequence between the incubation pool 223 and the carrier filter pool 224. The fifth channel 227 is configured as a capillary channel, so that the liquid in the incubation pool 223 can smoothly flow to the mixed solution transfer channel 226 under the siphon action of the capillary channel. The mixed solution transfer channel 226 is configured as an arc-shaped channel, which facilitates the transfer of the liquid in the mixed solution transfer channel to next treatment pool.

As shown in FIG. 11, the carrier filter pool 224 includes a first membrane placement chamber 2241 and a mixed solution chamber 2242, the mixed solution chamber 2242 is provided at a side close to the rotation center 1 relative to the first membrane placement chamber 2241. The first membrane placement chamber 2241 has a greater depth than the mixed solution chamber 2242, so that the liquid in the mixed solution chamber 2242 can enter the first membrane placement chamber 2241 under the action of gravity.

As shown in FIG. 12, a first filter assembly 2243 is inserted in the first membrane placement chamber 2241, and the first filter assembly 2243 includes a first membrane base plate 22431 for supporting the surface-modified membrane 22432 and the surface-modified membrane 22432. The first membrane base plate 22431 is hollow in the middle, and the surface-modified membrane 22432 is provided at a side of the first membrane base plate 22431 close to the rotation center 1. After both the loading solution and the sample from the sample quantification pool 212 enter the incubation pool 223, the loading solution and the sample are mixed in the incubation pool 223, and the resulting mixed solution then enters the mixed solution chamber 2242 of the carrier filter pool 224, and then passes through the surface-modified membrane 22432 provided in the first membrane placement chamber 2241. In this way, the substances in the biological sample, including the extracellular secretion membrane structure, are trapped on the surface-modified membrane 22432, and others pass through the surface-modified membrane 22432 and then flow out from a hollow portion of the first membrane base plate 22431.

The side of the carrier filter pool 224 away from the rotation center 1 is configured in a step-like manner and this arrangement facilitates the transfer of liquid from a previous treatment pool to next treatment pool.

The downstream side of the carrier filter pool 224 is connected to the enrichment unit 23; the upstream side of the carrier filter pool 224 is connected to the eluent pool 222.

The eluent pool 222 is provided on the side of the carrier filter pool 224 close to the rotation center 1, and the eluent pool 222 is configured as a square pool. As shown in FIG. 11, an eighth channel 228 is provided between the eluent pool 222 and the carrier filter pool 224 to connect the eluent pool 222 and the carrier filter pool 224.

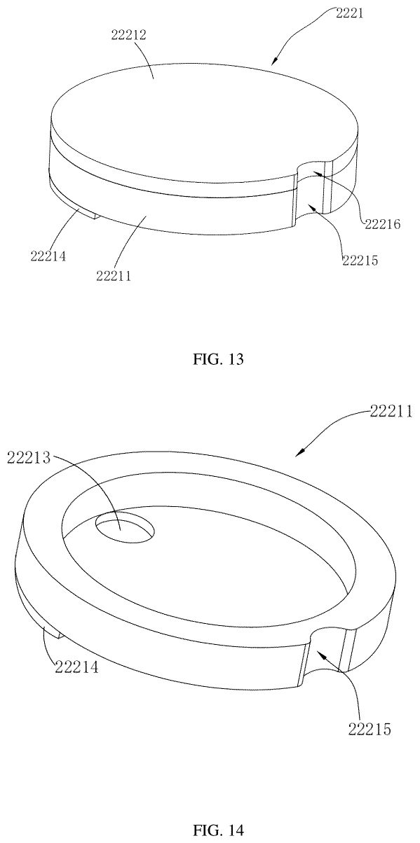

As shown in FIG. 13, The eluent pool 222 includes an eluent container assembly 2221 provided in the eluent pool 222; the eluent container assembly 2221 includes an eluent container 22211 and a first container lid 22212, and the first container lid 22212 matches the eluent container 22211 in shape; when the eluent container 22211 and the first container lid 22212 are placed in the eluent pool 222, an upper surface of the first container lid 22212 and an upper surface of the eluent pool 222 are substantially flush.

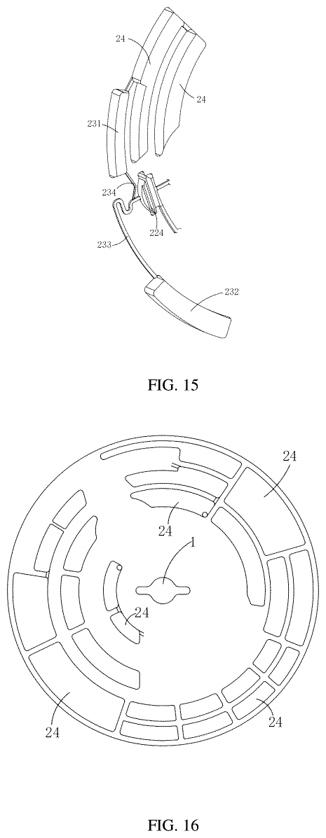

As shown in FIG. 11, a first ejector pin 2222 is provided at the bottom of the eluent pool 222 close to the eighth channel 228, a ninth channel 22221 is provided on an upper surface of the first ejector pin 2222 and points to the eighth channel 228. A first support platform 2223 is integrally formed on the bottom of the eluent pool 222 at a position away from the first ejector pin 2222 hole; as shown in FIG. 14, a first liquid release hole 22213 is provided at the bottom of the eluent container 22211, and a first sealing film 22214 is also adhered to the bottom of the eluent container 22211 to seal the first ejector pin 2222 hole. The material of the first sealing film 22214 is the same as that of the second sealing film 22114. During extraction, when the first ejector pin 2222 pushes the eluent container 22211 by means of the first liquid release hole 22213, the first support platform 2223 serves as the fulcrum of the eluent container 22211 to take a leverage effect so that the first ejector pin 2222 breaks the first sealing film 22214 on the eluent container 22211 to release the eluent. This solution can realize the isolation and extraction of the target extract from the biological sample in one go.

In addition, a first bump 2224 is provided on a side wall of the eluent pool 222, and a first recessed wall 22215 is provided on an outer side of the eluent container 22211, and a first notch 22216 that matches the first bump 2224 is formed at a corresponding position on the first container lid 22212. This arrangement ensures that the eluent container assembly 2221 can be firmly fixed in the eluent pool 222.

As shown in FIG. 15, then enrichment unit 23 includes a target extract pool 231 and a waste liquid pool 232 that are respectively connected to the carrier filter pool 224. The target extract pool 231 is configured to collect the target extract from the carrier filter pool 224, and the waste liquid pool 232 is configured to collect waste liquid from the carrier filter pool 224.

A sixth channel 233 is provided between the carrier filter pool 224 and the waste liquid pool 232, so that the waste liquid flowing out of the carrier filter pool 224 enters the waste liquid pool 232 via the sixth channel 233. The sixth channel 233 is configured as a curved channel, and the arrangement of the curved channel is conducive to increasing the path of the channel. When the mixture from the carrier filter pool 224 passes through the surface-modified membrane 22432, the extracellular secretory membrane structure in the biological sample is trapped on the surface-modified membrane 22432, and the waste liquid enters the waste liquid pool 232 via the sixth channel 233.

A seventh channel 234 is provided between the carrier filter pool 224 and the target extract pool 231, so that the target extract flowing out of the carrier filter pool 224 enters the target extract pool 231 via the seventh channel 234. A liquid inlet end of the seventh channel 234 is connected to the sixth channel 233. Connected ends of the seventh channel 234 and the sixth channel 233 are arranged at an acute angle of 60° and this arrangement facilitates the transfer of the target extract to the target extract pool 231. In addition, when the seventh channel 234 and the sixth channel 233 are arranged at an acute angle or a right angle and the sixth channel 233 is configured as a curved channel, less eluent containing the target extract enters the waste liquid pool 232, and the loss of the target extract can be reduced.

Further, the target extract pool 231 and one weight-reduction balancing pool 24 are connected by an air channel, and the cover plate 4 is provided with an air hole 43 at a corresponding position.

As shown in FIG. 3, the rotation center 1, ejector pin holes and the air holes 43 are provided at corresponding positions on the cover plate 4. That is, a first ejector pin hole 41 is provided on the cover plate 4 at a position corresponding to the first ejector pin 2222, and a second ejector pin hole 42 is provided on the cover plate 4 at a position corresponding to the second ejector pin 2212. In addition, as shown in FIG. 16, the bottom plate 3 is provided with a plurality of weight-reduction balancing pools 24, and the plurality of weight-reduction balancing pools 24 are provided around the rotation center 1 on the bottom plate 3 along the circumferential direction.

The main body of the isolation device is made from polycarbonate (PC) which is a polymer material, that is, the bottom plate 3 and the cover plate 4 are made from PC.

The bottom plate 3 and the cover plate 4 are bonded by heat pressure. The eluent container 22211 and the first container lid 22212 are bonded by heat pressure. The loading solution container 22111 and the second container lid 22112 are bonded by heat pressure. The eluent container and the loading solution container 22111 are made from a hydrophobic material. The bottom plate 3 has a thickness of 6 mm.

A method for extracting an extracellular secretory membrane structure includes the following steps:
I. 500 μL of whole blood sample was added to the sample pool 211 and the isolation and extraction device was controlled to rotate rapidly at 4500 rpm for 150 s, so that the sample quantification pool 212 was full filled with the biological example (the volume of the sample quantification pool 212 was 120 μL), and at the same time, some impurities entered the first impurity collection pool 216 to obtain plasma;
II. the plasma was allowed to stand still for 30 s, during which the plasma in the sample quantification pool 212 gradually entered the impurity filter pool 251 under the siphon action of the eleventh channel 252 and at the same time, the second ejector pin 2212 broke the second sealing film 22114, causing the loading solution (the volume of the loading solution pre-embedded was 120 μL) to flow out of the loading solution pool 221;
III. the isolation and extraction device was controlled to rotate rapidly at 2500 rpm for 20 s, so that the plasma entering the impurity filter pool 251 passed through the microfiltration membrane 25132, large-sized impurities were trapped by the microfiltration membrane 25132 and the rest entered the incubation pool 223 and at the same time, the loading solution flowing out of the loading solution pool 221 also entered the incubation pool 223, and the biological sample and the loading solution were mixed in the incubation pool 223;
IV. the isolation and extraction device was controlled to rotate in a high-low-high speed cycling pattern to promote the accelerated mixing of the two solutions in the incubation pool 223; the isolation and extraction device was first controlled to rotate rapidly at 3000 rpm for 5 s, then at 500 rpm for 5 s, and then at 3000 rpm for 5 s, and the isolation and extraction device was controlled to rotate in this high-low-high speed pattern for 10 cycles.
V. the isolation and extraction device was stopped to allow the mixed solution to stand still for 30 s so that the fifth channel 227 was full filled with the mixed solution;
VI. the isolation and extraction device was controlled to rotate rapidly at 3000 rpm for 60 s, so that the mixed solution entered the carrier filter pool 224 and passed through the surface-modified membrane 22432; consequently, the exosome in the mixed solution was trapped on the surface-modified membrane 22432 and the waste liquid entered the waste liquid pool 232;
VII. the isolation and extraction device was stopped to allow the solution to stand still for 30 s, during which the first ejector pin 2222 broke the first sealing film 22214, causing the eluent (the volume of the eluent solution pre-embedded was 250 μL) to flow out of the eluent pool 222; and
VIII. the isolation and extraction device was controlled to rotate rapidly at 5000 rpm for 50 s, so that the eluent, after passing through the surface-modified membrane 22432, eluted the extracellular secretory membrane structure from the surface-modified membrane 22432, and then entered the target extract pool 231.

An isolation and extraction kit includes the isolation and extraction device described above. The separation and extraction device is provided therein with a surface-modified membrane and a microfiltration membrane and is loaded with 120 μL of the loading solution and 250 μL of the eluent.

### Example 7

This example is the same as Example 6 except that the surface-modified membrane used in this example was prepared in accordance with the Preparation Example 7 of the surface-modified membrane and was loaded with QAS_{C18}; and that a method for extracting an extracellular secretory membrane structure includes the following steps:
I. 350 μL of whole blood sample was added to the sample pool 211 and the isolation and extraction device was controlled to rotate rapidly at 3500 rpm for 180 s, so that the sample quantification pool 212 was full filled with the plasma sample (the volume of the sample quantification pool 212 was 120 μL) and at the same time, some impurities entered the first impurity collection pool 216;
II. the plasma sample was allowed to stand still for 40 s, during which the plasma sample gradually entered the impurity filter pool 251 under the siphon action of the eleventh channel 252 and at the same time, the second ejector pin 2212 broke the second sealing film 22114, causing the loading solution (the volume of the loading solution pre-embedded was 120 μL) to flow out of the loading solution pool 221;
III. the isolation and extraction device was controlled to rotate rapidly at 3500 rpm for 30 s, so that the plasma sample entering the impurity filter pool 251 passed through the microfiltration membrane 25132, some impurities with large particle size and large molecular weight were trapped by the microfiltration membrane 25132 and the rest entered the incubation pool 223 and at the same time, the loading solution flowing out of the loading solution pool 221 also entered the incubation pool 223, and the biological sample and the loading solution were mixed in the incubation pool 223;
IV. the isolation and extraction device was controlled to rotate in a high-low-high speed cycling pattern to promote the accelerated mixing of the two solutions in the incubation pool 223; the isolation and extraction device was first controlled to rotate rapidly at 2500 rpm for 8 s, then at 300 rpm for 8 s, and then at 2500 rpm for 8 s, and the isolation and extraction device was controlled to rotate in this high-low-high speed pattern for 5 cycles;
V. the isolation and extraction device was stopped to allow the mixed solution stand still for 40 s so that the fifth channel 227 was full filled with the mixed solution;
VI. the isolation and extraction device was controlled to rotate rapidly at 2500 rpm for 40 s, so that the mixed solution entered the carrier filter pool 224 and passed through the surface-modified membrane 22432; consequently, the exosome in the mixed solution was trapped on the surface-modified membrane 22432 and the waste liquid entered the waste liquid pool 232;
VII. the isolation and extraction device was stopped to allow the solution to stand still for 40 s, during which the first ejector pin 2222 broke the first sealing film 22214, causing the eluent (the volume of the eluent solution pre-embedded was 200 μL) to flow out of the eluent pool 222; and
VIII. the isolation and extraction device was controlled to rotate rapidly at 3500 rpm for 50 s, so that the eluent, after passing through the surface-modified membrane 22432, eluted the exosome from the surface-modified membrane 22432, and then entered the target extract pool 231.

### Example 8

A method for extracting an exosome, implemented using syringes, is provided, where two syringes 6 are used and they are a first syringe and a second syringe, respectively. A surface-modified membrane is fixed in the first syringe at one end close to a needle-holding end; a microfiltration membrane is fixed in the second syringe at one end close to a needle-holding end. The volumes of the first syringe and the second syringe are both 3 mL.

The extraction method includes the following steps.

In step S1, 500 μL of whole blood sample was added to the second syringe. A piston rod of the second syringe was pressed to allow the whole blood sample to pass through the microfiltration membrane. The filtrate was taken for later use to obtain plasma.

In step S2, the plasma (200 μL) and the loading solution (200 μL) were mixed at a volume ratio of 1:1, and the resulting mixed solution was then placed in the first syringe provided therein with the surface-modified membrane. The surface-modified membrane used in this example was prepared in accordance with the Preparation Example 8 of the surface-modified membrane and was loaded with cocamidopropyl betaine. Then, the mixed solution was allowed to stand still until all the mixed solution passed through the surface-modified membrane and the filtrate was discarded.

In step S3, an eluent (300 μL) was then added to the first syringe and a piston rod of the first syringe was pressed to push the eluent to elute the surface-modified membrane loaded with the exosome. The high-concentration salt solution in the eluate caused the exosome to detach from the surface-modified membrane to obtain an exosome collection fluid.

The main components of the loading solution used were 100 mM tris-propane and 170 mM NaCl, pH 6.5. The main components of the eluent used were 50 mM tris-propane and 2.5 M NaCl, pH 6.36.

### Example 9

This example is the same as Example 8 except that the surface-modified membrane used in this example was prepared in accordance with the Preparation Example 9 of the surface-modified membrane and was loaded with octyldecylamidopropyl betaine.

### Example 10

This example is the same as Example 6 except that the surface-modified membrane used in this example was prepared in accordance with the Preparation Example 10 of the surface-modified membrane and was loaded with SPPT; and that
a method for extracting exosomes includes the following steps:
   I. 400 μL of whole blood sample was added to the sample pool 211 and the isolation and extraction device was controlled to rotate rapidly at 3000 rpm for 180 s, so that the sample quantification pool 212 was full filled with the plasma sample (the volume of the sample quantification pool 212 was 120 μL) and at the same time, some impurities entered the first impurity collection pool 216;
   II. the plasma sample was allowed to stand still for 60 s, during which the plasma sample gradually entered the impurity filter pool 251 under the siphon action of the eleventh channel 252 and at the same time, the second ejector pin 2212 broke the second sealing film 22114, causing the loading solution (the volume of the loading solution pre-embedded was 120 μL) to flow out of the loading solution pool 221;
   III. the isolation and extraction device was controlled to rotate rapidly at 3000 rpm for 20 s, so that the plasma sample entering the impurity filter pool 251 passed through the microfiltration membrane 25132, some impurities with large particle size and large molecular weight were trapped by the microfiltration membrane 25132 and the rest entered the incubation pool 223 and at the same time, the loading solution flowing out of the loading solution pool 221 also entered the incubation pool 223, and the biological sample and the loading solution were mixed in the incubation pool 223;
   IV. the isolation and extraction device was controlled to rotate in a high-low-high speed cycling pattern to promote the accelerated mixing of the two solutions in the incubation pool 223; the isolation and extraction device was first controlled to rotate rapidly at 3000 rpm for 10 s, then at 800 rpm for 10 s, and then at 3000 rpm for 10 s, and the isolation and extraction device was controlled to rotate in this high-low-high speed pattern for 15 cycles;
   V. the isolation and extraction device was stopped to allow the mixed solution to stand still for 60 s so that the fifth channel 227 was full filled with the mixed solution;
   VI. the isolation and extraction device was controlled to rotate rapidly at 3000 rpm for 40 s, so that the mixed solution entered the carrier filter pool 224 and passed through the surface-modified membrane 22432; consequently, the exosome in the mixed solution was trapped on the surface-modified membrane 22432 and the waste liquid entered the waste liquid pool 232;
   VII. the isolation and extraction device was stopped to allow the solution to stand still for 60 s, during which the first ejector pin 2222 broke the first sealing film 22214, causing the eluent (the volume of the eluent solution pre-embedded was 300 μL) to flow out of the eluent pool 222; and
   VIII. the isolation and extraction device was controlled to rotate rapidly at 4000 rpm for 50 s, so that the eluent, after passing through the surface-modified membrane 22432, eluted the exosome from the surface-modified membrane 22432, and then entered the target extract pool 231.

### Example 11

This example is the same as Example 1 except that the surface-modified membrane used in this example was prepared in accordance with the Preparation Example 11 of the surface-modified membrane and was loaded with DTAB and cocamidopropyl betaine; and that
the main components of the loading solution used were 100 mM tris-propane and 170 mM NaCl, pH 6.5; and the main components of the eluent used were 50 mM tris-propane and 2.5 M NaCl, pH 6.36.

### Example 12

This example is the same as Example 6 except that the surface-modified membrane used in this example was prepared in accordance with the Preparation Example 12 of the surface-modified membrane and was loaded with QAS_{C0} and SPPT; and that
the main components of the loading solution used were 100 mM tris-propane and 170 mM NaCl, pH 6.5; and the main components of the eluent used were 50 mM tris-propane and 2.5 M NaCl, pH 6.36.

In addition, this example also differs from example 6 in the structure of the incubation pool of the microfluidic centrifuge disk. Specifically:

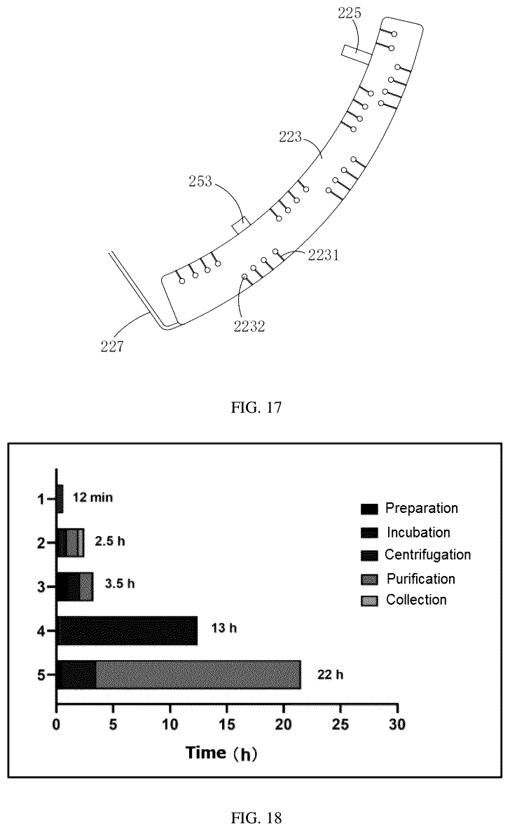

As shown in FIG. 17, the incubation pool 223 also includes a number of elastic rods 2231; one end of each elastic rod 2231 is fixedly connected to the side wall of the incubation pool 223, or may be integrally formed thereon; the other end of the elastic rod 2231 is integrally formed with an elastic ball 2232. Since the elastic rods 2231 and the elastic balls 2232 are provided, when the liquid flows and during centrifugation, the forces acting on the elastic rods 2231 and the elastic balls 2232 are inconsistent, causing the elastic balls 2232 and the elastic rods 2231 to drive the liquid to flow. So the elastic rods 2231 and the elastic balls 2232 can accelerate stirring so that the liquid in the incubation pool 223 is well mixed. In addition, no elastic rods 2231 and/or elastic balls 2232 are provided close to the fourth channel 225, the fifth channel 227, and the twelfth channel 253 to prevent the elastic ball 2232 and/or the elastic rod 2231 from clogging the channels.

### Example 13

This example is the same as Example 12 except that the surface-modified membrane used in this example was prepared in accordance with the Preparation Example 13 of the surface-modified membrane and was loaded with QAS_{C18} and SPPT; and that
the main components of the loading solution used were 100 mM tris-propane and 170 mM NaCl, pH 6.5; and the main components of the eluent used were 50 mM tris-propane and 2.5 M NaCl, pH 6.36.

### Example 14

This example is the same as Example 12 except that the surface-modified membrane used in this example was prepared in accordance with the Preparation Example 14 of the surface-modified membrane and was loaded with QAS_{C18}, SPPT and a betaine; and that
the main components of the loading solution used were 100 mM tris-propane and 170 mM NaCl, pH 6.5; and the main components of the eluent used were 50 mM tris-propane and 2.5 M NaCl, pH 6.36.

### Example 15

This example is the same as Example 12 except that the surface-modified membrane used in this example was prepared in accordance with the Preparation Example 15 of the surface-modified membrane and was loaded with QAS_{C0}, SPPT and proline; and that
the main components of the loading solution used were 100 mM tris-propane and 170 mM NaCl, pH 6.5; and the main components of the eluent used were 50 mM tris-propane and 2.5 M NaCl, pH 6.36.

### Example 16

This example is the same as Example 12 except that: the surface-modified membrane used in this example was prepared in accordance with the Preparation Example 16 of the surface-modified membrane and was loaded with QAS_{C0}, SPPT and mannitol; and
the main components of the loading solution used were 100 mM tris-propane and 170 mM NaCl, pH 6.5; and the main components of the eluent used were 50 mM tris-propane and 2.5 M NaCl, pH 6.36.

### Example 17

This example is the same as Example 12 except that the surface-modified membrane used in this example was prepared in accordance with the Preparation Example 17 of the surface-modified membrane and was loaded with QAS_{C0}, SPPT, a betaine and proline; and that
the main components of the loading solution used were 100 mM tris-propane and 170 mM NaCl, pH 6.5; and the main components of the eluent used were 50 mM tris-propane and 2.5 M NaCl, pH 6.36.

### Example 18

This example is the same as Example 12 except that the surface-modified membrane used in this example was prepared in accordance with the Preparation Example 18 of the surface-modified membrane and was loaded with QAS_{C0}, SPPT, a betaine, mannitol and proline; and that
the main components of the loading solution used were 100 mM tris-propane and 170 mM NaCl, pH 6.5; and the main components of the eluent used were 50 mM tris-propane and 2.5 M NaCl, pH 6.36.

### Example 19

This example is the same as Example 18 except that the surface-modified membrane used in this example was prepared in accordance with the Preparation Example 19 of the surface-modified membrane and was loaded with QAS_{C18}, SPPT, a betaine, mannitol and proline.

### Example 20

This example is the same as Example 18 except that the surface-modified membrane used in this example was prepared in accordance with the Preparation Example 20 of the surface-modified membrane and was loaded with cocamidopropyl betaine, a betaine, mannitol and proline.

### Example 21

This example is the same as Example 18 except that the surface-modified membrane used in this example was prepared in accordance with the Preparation Example 21 of the surface-modified membrane and was loaded with SPPT, a betaine, mannitol and proline.

### Example 22

This example is the same as Example 10 except that the treated whole blood sample in step S1 was replaced with an equal volume of cell supernatant.

In this solution, the cell that can be selected includes but is not limited to any one of A549 cell, H1299 cell, H23 cell and Beas-2b cell. In this example, the cell used was A549 cell which is commonly commercially available.

Step S1 was specifically implemented as follows:
Obtaining of cell supernatant: the obtained A549 cell was cultured by an existing method to obtain cell culture fluid, and the cell culture fluid was then centrifuged at 3000 g for 25 min to obtain initial cell supernatant; then, 40 mL of the initial cell supernatant was taken and pre-concentrated to the same volume as the whole blood sample to obtain the cell supernatant.

The cell supernatant, the loading solution and the eluent were placed at the corresponding positions on the microfluidic centrifuge disk.

### Example 23

This example is the same as Example 10 except that the treated whole blood sample in step S1 was replaced with an equal volume of hydrothorax and ascites. Step S1 was specifically implemented as follows:
Obtaining of hydrothorax and ascites: a sample from a patient of Maoming People's Hospital; 20 mL of initial hydrothorax and ascites was collected from the patient, and then pre-concentrated to the same volume as the whole blood sample to obtain hydrothorax and ascites.

The hydrothorax and ascites, the loading solution and the eluent were placed at the corresponding positions on the microfluidic centrifuge disk.

### Example 24

This example is the same as Example 10 except that the treated whole blood sample in step S1 was replaced with an equal volume of urine. Step S1 was specifically implemented as follows:
Obtaining of urine: 20 mL of initial urine and then pre-concentrated to the same volume as the whole blood sample to obtain urine.

The urine, the loading solution and the eluent were placed at the corresponding positions on the microfluidic centrifuge disk.

### Example 25

This example is the same as Example 10 except that the treated whole blood sample in step S1 was replaced with an equal volume of plasma. Step S1 was specifically implemented as follows:
Obtaining of plasma: whole blood was centrifuged at 3000 rpm for 200 s to obtain plasma.

The plasma, the loading solution and the eluent were placed at the corresponding positions on the microfluidic centrifuge disk.

### Example 26

This example is the same as Example 10 except that the treated whole blood sample in step S1 was replaced with an equal volume of saliva. Step SI was specifically implemented as follows:
Obtaining of saliva: 20 mL of initial urine and then pre-concentrated to the same volume as the whole blood sample to obtain saliva.

The saliva, the loading solution and the eluent were placed at the corresponding positions on the microfluidic centrifuge disk.

### Comparative Example 1

Extraction of an exosome by size exclusion chromatography includes the following steps.

Size exclusion chromatography:
I. preparation of a separation column and a sample
preparation of a separation column: (1) a size exclusion separation column (purchased from Shanghai Wayen Biotechnologies, product model: qEVoriginal/35nm) was subjected to column equilibration at room temperature;
(2) a PBS buffer was loaded to the separation column from step (1) and column equilibration was performed;
preparation of a sample: (1) 1 mL of whole blood was centrifuged at 1500 g for 10 min and the resulting supernatant was collected;
(2) the supernatant from step (1) was centrifuged at 2000 g for 10 min and the resulting supernatant was then collected;
(3) the supernatant from step (2) was filtered with a 0.22 um filter membrane to obtain a filtrate;
II. all the filtrate from step (3) was loaded to the separation column and the effluent was collected; and
III. 2.5 mL of a buffer was loaded to the separation column and the effluent was collected.

The first 3 mL of the effluents collected in step II and step III was waste liquid, and the following 2.5 mL of the effluents was collected as an exosome collection fluid.

### Comparative Example 2

Extraction of an exosome by ultracentrifugation includes the following steps:
I. Treatment of whole blood samples
(1) 1 mL of whole blood sample was centrifuged at 220 g and 4° C. for 10 min and the resulting pernatant was collected;
(2) the supernatant from step (1) was centrifuged at 2000 g and 4° C. for 20 min and the resulting supernatant was collected;
(3) the supernatant from step (2) was centrifuged at 15000 g and 4° C. for 30 min and the resulting supernatant was collected; and
(4) the supernatant from step (3) was filtered with a 0.22 μm syringe filter to remove particles with a diameter of more than 220 nm to obtain a filtrate.
II. All the supernatant obtained in step (4) of step I was transferred to an ultracentrifuge tube and centrifuged at 120,000 g and 4° C. for 70 min to obtain pellet.
III. The exosome pellet from step II was resuspended with an appropriate amount of PBS (about 100 μL) and step II was performed again.
IV. The exosome pellet from step III was resuspended with an appropriate amount of PBS (100 μL), thus obtaining the final extract.

### Comparative Example 3

Extraction of an exosome by PEG precipitation: an exosome extraction kit purchased from Geneseed Biotech was used to extract exosomes according to the operating procedures of the kit.

### Comparative Example 4

Extraction of an exosome by density gradient centrifugation includes the following steps:
I. Pretreatment of whole blood samples:
(1) 1 mL of whole blood sample was centrifuged at 2000 g for 10 min and the resulting supernatant was collected;
(2) the supernatant from step (1) was filtered with a 0.22 μm filter to obtain a filtrate.
II. 2 mL of 60 wt % iodixanol was added to the bottom of an ultracentrifuge tube and the filtrate from step (2) was then added; the resulting solution was then centrifuged at 10000 g and 4° C. for 3 h, the supernatant was discarded, and 3 mL of the filtrate at the bottom of the centrifuge tube was retained.
III. 2 mL of 20 wt %, 10 wt % and 5 wt % iodixanol was added in sequence to the filtrate from step II and the resulting solution was then centrifuged at 10000 g and 4° C. for 18 h, thus obtaining 7 mL of the final extract, i.e., the exosome collection fluid.

### Performance Testing

### (I) Time Distribution of Different Extraction Stages of the Extraction Methods

The time distribution of different stages of exosome extraction by the methods described in Example 10 and Comparative Examples 1-4 was recorded. See FIG. 18 for details. As shown in FIG. 18, the method of the present application for exosome extraction has a significant advantage in extraction time, which was only 12 min, much less than the extraction time of 2.5-22 h of the other four extraction methods.

### (II) Western Blot Test for Exosomes

Western blotting was performed using an SDS-PAGE gel preparation kit (commonly available). Protein lysates were resolved by SDS-PAGE and transferred to a polyvinylidene fluoride membrane. The polyvinylidene fluoride membrane was blocked at room temperature for 1 h and then incubated with a primary antibody overnight at 4° C.

The following antibodies were used for Western blot analysis: anti-CD63 (Abcam), anti-CD81 (Abcam), anti-CD9 (Abcam), anti-Alix (Abcam), anti-TSG101 (Abcam), and anti-GAPDH (Abcam). All primary antibodies were used at a dilution of 1:1000.

The polyvinylidene fluoride membrane was washed three times with TBST (1×TBS and 0.5 wt % Tween-20, pH7.4), then immersed in HRP-conjugated anti-rabbit IgG serving as a secondary antibody (1:3000) at room temperature, incubated for 60 min, and then immunodetected using enhanced chemiluminescence (Pall).

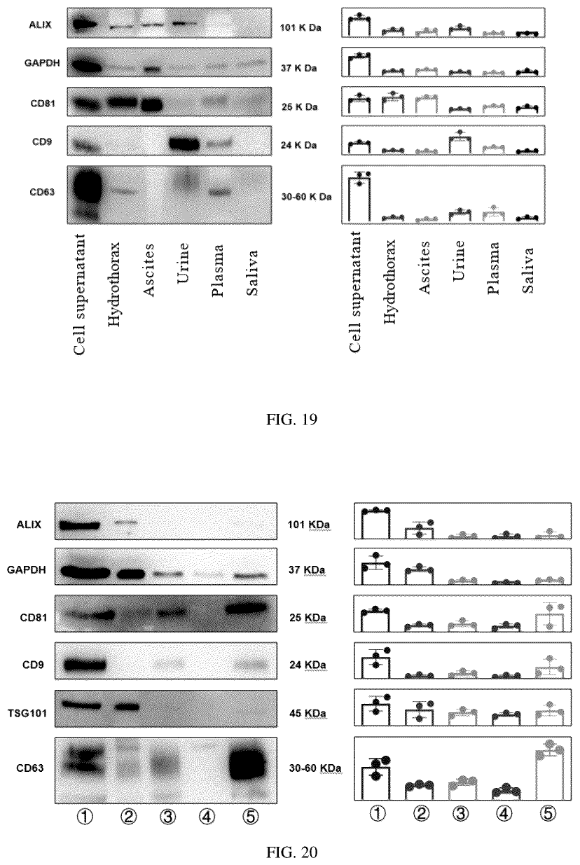

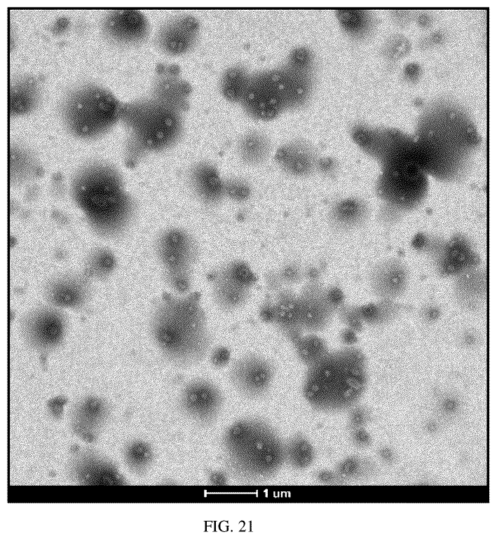

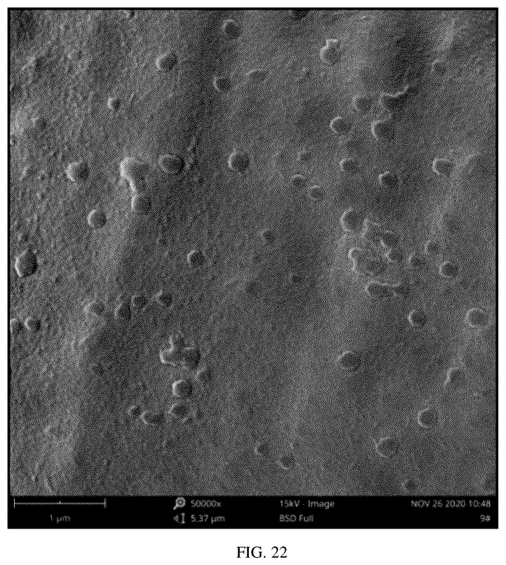

The exosome collection fluids of Example 10 and Examples 22-26 were tested, and the test results are shown in FIG. 19. The exosome collection fluids of Comparative Examples 1-4 were tested, and the test results are shown in FIG. 20. The results of FIG. 19 show that the extraction method of the present application can be used to extract exosomes from different biological samples. The results of FIG. 20 show that, when the method of the present application was used for extracting exosomes from biological samples, the protein concentrations of Alix, GAPDH, CD81, CD9, and CD63 obtained were relatively high, and the concentrations of these proteins were significantly higher than the extraction results of size exclusion chromatography, ultracentrifugation method, and PEG precipitation method.

### (III) Transmission Electron Microscopy (TEM) Detection for Exosomes

The transmission electron microscopy detection method for exosomes was performed as follows: 10 μL of exosome suspension was dropwise added onto a clean sealing film; a copper mesh with a carbon film was placed on the exosome suspension for 90 s and then picked up and the excess liquid was absorbed with filter paper from the side, and then the detection was performed. The exosomes obtained in Example 10 were detected by transmission electron microscopy, and the detection results are shown in FIG. 21.

### (IV) Scanning Electron Microscopy (SEM) Detection

The exosomes obtained in Example 10 were commissioned to be detected by SEM by Phenom Scientific Instruments Shanghai Co., Ltd. The results are shown in FIG. 22. The results show that the exosomes prepared in this example are particles with size distribution of about 100 nm.

### (V) Size Range Determination for Exosomes

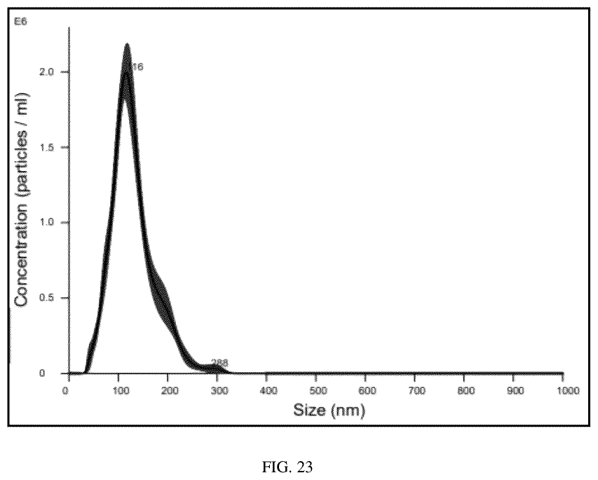

The exosomes obtained in Example 10 were commissioned to be determined by Southern Medical University for size range, and the results are shown in FIG. 23. The results show that the sizes of the exosomes prepared in this example are distributed within the size distribution range (about 100 nm) of exosomes, and the concentration of the exosomes is 10⁶ particles/mL.

The particle size range and concentration of the exosomes prepared in Examples 1-9 and 11-21 were determined, respectively, and the results are shown in Table 1.

TABLE 1
Particle size range and concentration of the exosomes prepared

| Example | Example 1 | Example 2 | Example 3 | Example 4 | Example 5 | Example 6 | Example 7 |
|---|---|---|---|---|---|---|---|
| Particle size (nm) | 180 | 180 | 130 | 120 | 120 | 110 | 110 |
| Concentration | 10⁴ | 10⁴ | 10⁴ | 10⁵ | 10⁵ | 10⁶ | 10⁶ |
| (particles/mL) | | | | | | | |
| Example | Example 8 | Example 9 | Example 10 | Example 11 | Example 12 | Example 13 | Example 14 |
| Particle size (nm) | 120 | 120 | 100 | 150 | 95 | 95 | 95 |
| Concentration | 10⁴ | 10⁴ | 10⁶ | 10⁵ | 10⁷ | 10⁷ | 10⁸ |
| (particles/mL) | | | | | | | |
| Example | Example 15 | Example 16 | Example 17 | Example 18 | Example 19 | Example 20 | Example 21 |
| Particle size (nm) | 95 | 95 | 95 | 95 | 95 | 120 | 110 |
| Concentration | 10⁸ | 10⁸ | 10⁸ | 10⁸ | 10⁸ | 10⁵ | 10⁷ |
| (particles/mL) | | | | | | | |

Referring to the data results in Table 1, by comparing Examples 14-16 with Example 12, it shows that the protective agents used can better protect exosomes, contributing to extraction of exosomes with a high concentration. This conclusion may also be drawn by comparison between Example 13 and Example 19, between Example 8 and Example 20, and between Example 10 and Example 21.

The specific examples are merely an explanation of the present application and not intended to limit the present application. Those skilled in the art can make modifications, without creative contribution, to the examples as needed after reading this description. Any of the modifications made within the scope of the claims of the present application shall be protected by the Patent Law.

What is claimed is:

1. A surface-modified membrane for extracting an exosome, wherein the surface-modified membrane comprises a base membrane and a surfactant loaded on the base membrane, and the surfactant is one or more selected from a group consisting of trimethoxysilylpropyl trimethyl ammonium chloride, trimethoxysilylpropyl octadecyldimethyl ammonium chloride and 3-(N,N-dimethyldodecyl ammonium) propane sulfonate.

2. The surface-modified membrane for extracting an exosome according to claim 1, wherein the base membrane is a regenerated cellulose (RC) membrane.

3. A method for extracting an exosome by using the surface-modified membrane according to claim 1, comprising the following steps:
step 1: adding a whole blood sample onto a microfluidic centrifuge disk;
step 2: separating the whole blood sample to obtain plasma;
step 3: filtering the plasma with a microfiltration membrane to obtain filtered plasma;
step 4: mixing the filtered plasma with a loading solution to obtain a mixed solution;
step 5: filtering the mixed solution with the surface-modified membrane to trap the exosome on the surface-modified membrane; and
step 6: eluting the exosome from the surface-modified membrane to obtain a target exosome.

4. The method according to claim 3, wherein the microfluidic centrifuge disk comprises a rotation center and an extraction mechanism, the extraction mechanism comprises a sample injection unit, a capturing and releasing unit and an enrichment unit, the capturing and releasing unit is connected to the sample injection unit and located at a downstream side of the sample injection unit, and the enrichment unit is connected to the capturing and releasing unit and located at a downstream side of the capturing and releasing unit.

5. The method according to claim 4, wherein the surface-modified membrane is provided in the capturing and releasing unit.

6. The method according to claim 3, wherein the microfiltration membrane is a polyethersulfone (PES) hydrophilic filter membrane.

7. The method according to claim 3, wherein the loading solution comprises tris-propane and NaCl.

8. The method according to claim 3, wherein a volume ratio of the filtered plasma to the loading solution is within a range of 1:2 to 2:1.

9. A method for extracting an extracellular secretory membrane structure by using the surface-modified membrane according to claim 1, comprising the following steps:
S1, adding a biological sample into an isolation and extraction device and performing pretreatment;
S2, mixing the biological sample after the pretreatment with a loading solution to obtain a mixed solution, and then centrifuging the mixed solution and passing the mixed solution through the surface-modified membrane, so that the extracellular secretory membrane structure in the biological sample is captured on the surface-modified membrane; and
S3, eluting the surface-modified membrane with an eluent to obtain a collection fluid containing the extracellular secretory membrane structure.

10. The method according to claim 9, wherein the biological sample is selected from a group consisting of whole blood, plasma, serum, urine, saliva, hydrothorax and ascites, cerebrospinal fluid, cell culture supernatant, tears, semen, amniotic fluid, gastric juice, saliva, nasal discharge, bronchoalveolar lavage fluid, joint synovial fluid, bile, uterine mucus and feces.

11. The method according to claim 9, wherein the isolation and extraction device comprises a rotation center and at least one extraction mechanism; the at least one extraction mechanism comprises a sample injection unit, a capturing and releasing unit, and an enrichment unit; the sample injection unit is configured to input and store the biological sample; the surface-modified membrane is provided in the capturing and releasing unit, and the capturing and releasing unit is connected to the sample injection unit and located at a downstream side of the sample injection unit to capture or release a target extract in the biological sample; and the enrichment unit is connected to the capturing and releasing unit and located at a downstream side of the capturing and releasing unit to enrich the target extract released by the capturing and releasing unit.

12. The method according to claim 9, wherein the extracellular secretory membrane structure comprises one or two selected from a group consisting of an extracellular vesicle and an exosome.
